(19) United States
(12) Patent Application Publication
(10) Pub. No.: US 2018/0057859 A1
(43) Pub. Date: Mar. 1, 2018

(54) METHOD FOR IDENTIFYING RARE CELL TYPES BY SINGLE CELL ASSISTED DECONVOLUTION OF POPULATION GENE EXPRESSION DATA

(71) Applicants: Craig E. NELSON, Mansfield Center, CT (US); Ion MANDOIU, Storrs, CT (US); James R. LINDSAY, Brookline, MA (US)

(72) Inventors: Craig E. NELSON, Mansfield Center, CT (US); Ion MANDOIU, Storrs, CT (US); James R. LINDSAY, Brookline, MA (US)

(21) Appl. No.: 15/589,535

(22) Filed: May 8, 2017

Related U.S. Application Data
(60) Provisional application No. 62/332,645, filed on May 6, 2016.

(51) Int. Cl. C12Q 1/68

(52) U.S. Cl. CPC C12Q 1/6809; C12Q 1/6869; C12Q 1/6851

(57) ABSTRACT

The invention provides methods of identifying the gene expression profile of a cell type present within a population of cells using single cell expression data by a residual minimization process. The methods of the invention can be used to infer the genes expressed by a rare cell type that can be difficult to measure experimentally due to its low abundance in an organism or sample of interest. The methods described herein can additionally be used to predicting the gene expression profile of a population of cells using single cell expression patterns as well as methods of quantitating the composition of a population.

### FIELD OF THE INVENTION

The invention relates to methods of identifying gene expression patterns of cell types present within biological samples and methods of determining the cell type composition of multi-cell populations.

### BACKGROUND OF THE INVENTION

One application of single cell genetics is the detection of rare cell types. Due to their low quantity, rare cells (such as progenitor cells or pluripotent stem cells) may only be present at low abundance in a sample, such as a tissue culture sample or a sample isolated from an organism. Examples of rare cells include those that have only transient gene expression patterns that are a subtle response to changes in environmental conditions. The likelihood that these rare cells are sampled in single cell experiments is proportional to the frequency with which these cell types appear in the population from which the sample is isolated. Due to their low frequency, the experimental observation of the gene expression profiles of rare cell types has been challenging. There exists a need for method of computationally inferring the canonical gene signature of rare cell types that often cannot be directly measured.

### SUMMARY OF THE INVENTION

The invention provides methods of determining the gene expression profile of a rare or otherwise difficult-to-sample cell type (e.g., a progenitor cell, stem cell, transient cell type, or disease cell), as well as methods of determining the composition of a population of cells that is expressed in terms of the types of cells present within the population. The methods described herein utilize single cell gene profiling analysis to determine gene expression patterns of distinct types of cells, which can in turn be extrapolated to determine predicted gene expression profiles of entire populations of cells. These predictions can then be compared with empirical measurements of the gene expression profile of the same populations, such as experimentally determined genetic or protein profiles observed by, e.g., polymerase chain reaction (PCR), RNA sequencing (RNA-Seq), enzyme-linked immunosorbent assay (ELISA), or other methods as described herein. Minimization of the residual between the predicted and empirical gene expression profiles results in the determination of the gene expression profile of the missing cell present within the population but not sampled and observed at the level of a single cell.

In a first aspect, the invention features a method of determining the gene expression profile of a rare cell type in a population of cells. The method includes the steps of:
a. providing gene expression profiles of a plurality of individual cells in the population;
b. using the gene expression profiles provided in (a) to determine a predicted gene expression profile of the entirety of the population; and
c. minimizing the difference between the predicted gene expression profile determined in (b) and an experimentally determined gene expression profile of the entirety of the population.

The minimizing thus determines the gene expression profile of the rare cell type.

In some embodiments, the predicted gene expression profile of the entirety of the population is determined using predicted gene expression profiles of a plurality of cell types present within the population and a calculated cell type composition of the population. In some embodiments, the predicted gene expression profiles of the plurality of cell types present within the population are determined by grouping the gene expression profiles of the plurality of individual cells into like clusters. For example, the grouping may include performing principle component analysis (PCA). In some embodiments, the grouping includes performing K-means clustering.

In some embodiments, the difference between the experimentally determined gene expression profile of the entirety of the population of cells and the predicted gene expression profile of the entirety of the population of cells is minimized by least-squares optimization.

The gene expression profiles of the plurality of individual cells in the population may be determined by measuring the abundance of one or more RNA transcripts, or DNA equivalents thereof, in each of the individual cells. Similarly, the experimentally determined gene expression profile of the entirety of the population of cells may be determined by measuring the abundance of one or more RNA transcripts, or DNA equivalents thereof, in the entirety of the population.

In some embodiments, the abundance of the one or more RNA transcripts, or the DNA equivalents thereof, is measured using a PCR assay. For example, the PCR assay may be a quantitative reverse transcription PCR (qRT-PCR) assay. In some embodiments, the PCR assay is conducted by contacting DNA or RNA isolated from the individual cells with an oligonucleotide complementary to a portion of a gene of interest. The oligonucleotide may contain, e.g., a radioisotope, fluorescent compound, bioluminescent compound, a chemiluminescent compound, metal chelator, or enzyme. In some embodiments, the oligonucleotide contains a fluorescent probe, and may optionally include a fluorescence quencher. In some embodiments, the abundance of the one or more RNA transcripts is measured using a RNA-Seq assay.

The gene expression profiles of the plurality of individual cells in the population may be determined by measuring the abundance of one or more proteins in the individual cells. Similarly, the experimentally determined gene expression profile of the entirety of the population of cells may be determined by measuring the abundance of one or more proteins in the entirety of the population. In some embodiments, the abundance of the one or more proteins is measured using an immunohistochemical assay, a western blot analysis, immunoprecipitation, an enzyme-linked immunosorbent assay (ELISA), an enzyme-linked immunofiltration assay (ELIFA), a molecular binding assay, mass spectrometry, mass spectrometric immunoassay, or a biochemical enzymatic activity assay.

In some embodiments, the rare cell type is present within the population of cells at low abundance, e.g., as described herein. For example, the rare cell type may be present within the population of cells at concentrations below the limit of detection of a gene expression assay, such as PCR, qRT-PCR, RNA-Seq, an immunohistochemical assay, a western blot analysis, immunoprecipitation, ELISA, ELIFA, a molecular binding assay, mass spectrometry, mass spectrometric immunoassay, and a biochemical enzymatic activity assay.

In some embodiments, the rare cell type includes a component of a mixture of cells.

In some embodiments, the rare cell type includes adult cells and/or cancer cells. In certain embodiments, the cancer cells include circulating tumor cells and/or rare cancer cells.

In some embodiments, cells of the rare cell type (e.g., rare cancer cells) are present in a tissue biopsy.

In some embodiments, the rare cell type is a hematopoietic, intestinal, mammary gland, prostate, or neural stem cell.

In another aspect, the invention provides a method of identifying the cell type composition of a population of cells. The method includes the steps of:
a. providing gene expression profiles of a plurality of individual cells in the population;
b. using the gene expression profiles provided in (a) to determine predicted gene expression profiles of a plurality of cell types present within the population by grouping the gene expression profiles of the plurality of individual cells into like clusters; and
c. comparing the gene expression profiles determined in (b) with the gene expression profile of the entirety of the population of cells;
wherein the comparing identifies the cell type composition of the population of cells.

### Definitions

As used herein, the term “cell type” refers to a group of cells sharing a phenotype that is statistically separable based on gene expression data. For instance, cells of a common cell type may share similar structural and/or functional characteristics, such as similar gene activation patterns and antigen presentation profiles. Cells of a common cell type may include those that are isolated from a common tissue (e.g., epithelial tissue, neural tissue, connective tissue, or muscle tissue) and/or those that are isolated from a common organ, tissue system, blood vessel, or other structure and/or region in an organism. Exemplary methods and compositions for defining a cell type are described, for example, in PCT Publication No. WO 1999/029877 and U.S. Pat. No. 6,110,711, each of which is incorporated herein by reference with respect to such methods and compositions.

As used herein, the term “cell type composition” refers to the distribution of cell types present within a population of cells. In some instances, a cell type composition may take the form of a matrix (i.e., a “concentration matrix”) to display, for example, the quantity or relative abundance of cell types present within one or more populations of cells. In the case of homogeneous populations, for instance, the cell type composition contains information regarding the quantity or relative abundance of the single cell type present within each population. In the case of heterogeneous populations, the cell type composition contains information regarding the quantity or relative abundance of each of the various cell types present within each population. Exemplary methods and systems for determining the cell type composition of a mixed cell population are described, for example, in PCT Publication No. WO 2005/028677, incorporated herein by reference with respect to such methods and systems.

As used herein, the term “endogenous” describes a molecule (e.g., a polypeptide, nucleic acid, or cofactor) or complex that is found naturally in a particular organism (e.g., a human) or in a particular location within an organism (e.g., an organ, a tissue, or a cell, such as a human cell).

As used herein, the term “exogenous” describes a molecule (e.g., a polypeptide, nucleic acid, or cofactor) that is not found naturally in a particular organism (e.g., a human) or in a particular location within an organism (e.g., an organ, a tissue, or a cell, such as a human cell). Exogenous materials include those that are provided from an external source to an organism or to cultured matter extracted therefrom.

As used herein, the term “gene expression profile” refers to a measurement of the level of one or more genes expressed by an individual cell, a cell type, or by a plurality of cells present in a homogeneous or heterogeneous mixture of cell types. The expression level of a gene expressed by a single cell, type of cell, or plurality of cells can be ascertained, for example, by inferring the quantity or concentration of RNA transcripts, such as mRNA, or a DNA equivalent thereof, present in a sample that correspond to the gene of interest. Determining the abundance of RNA transcripts, or DNA equivalents thereof, in a sample can be performed using established techniques known in the art, including quantitative, reverse-transcription polymerase chain reaction (qRT-PCR) assays and deep sequencing techniques, as well known in the art. Exemplary techniques useful for determining RNA transcript levels include RNA sequencing assays (RNA-Seq), e.g., as described herein. Gene expression levels can also be determined by measuring the quantity of protein produced from translation of such transcripts, for instance, using an enzyme-linked immunosorbent assay (ELISA) format in conjunction with one or more antibodies that specifically binds the protein of interest.

As used herein, the term “low abundance” refers to the level of gene expression that is relatively less, for example, than the average (e.g., mean or median or an approximation thereof) of detectable genes expressed in a library, for instance, using standard gene detection techniques known in the art, such as qPCR or RNA-Seq. In some instances, a gene expressed at “low abundance” is generally expressed at levels among the bottom half (e.g., bottom half, bottom third, bottom quarter, bottom 10%, bottom 5%, bottom 1%, bottom 0.1%, or lower) of the transcriptome or proteome.

As used herein, the term “population” when applied to cells refers to a collection of cells present within an organism or within a sample (e.g., cells isolated from an organism or progeny thereof). A population of cells may be homogeneous (i.e., consisting of cells of a single cell type) or heterogeneous (i.e., containing cells of two or more (e.g., 2, 3, 4, 5, 6, 7, 8, 9, 10, 20, 30, 40, 50, 60, 70, 80, 90, 100, or more) cell types).

As used herein, the term “RNA sequencing assay” or “RNA-Seq” refers to an assay that can be used to determine the presence and/or quantity of one or more RNA transcripts (e.g., hnRNA or alternatively spliced mRNA) within a sample of RNA molecules. Such RNA sequencing assays may include high-throughput sequencing methods as known in the art. Exemplary methods for conducting RNA-Seq assays are described herein.

As used herein, the term “sample” generally refers to a specimen (e.g., blood, blood component (e.g., serum or plasma), urine, saliva, amniotic fluid, cerebrospinal fluid, tissue (e.g., placental or dermal), pancreatic fluid, chorionic villus sample, and cells) isolated from and/or derived from an organism (e.g., a mammalian organism, such as a human, mouse, rat, canine, feline, or bovine animal. A sample may also include a collection of isolated single-celled organisms, such as a bacterial strain or single-celled eukaryote, for example, a single-cell fungus (e.g., a yeast strain) or a protist. A sample may be a primary sample isolated directly from an organism (e.g., a clinical sample) or may be a sample derived from such a primary sample (e.g., a pool of cDNAs produced from an RNA sample). A sample may be a clinical sample. Examples of such samples are well known in the art.

As used herein, the term “cluster” refers to a group of like data points, for example, that are grouped together based on the proximity of the data points to a measure of central tendency of the cluster. For instance, the measure of central tendency may be the arithmetic mean of the cluster, in which case the data points are joined together based on their proximity to the average value in the cluster (see, for example, “K-means clustering”). Clustering methods useful in conjunction with the methods of the invention are known in the art and are described herein.

As used herein, the term “K-means clustering” refers to a process of grouping like data sets (e.g., gene expression profiles) into groups (e.g., “clusters”) in which each data set belongs to the cluster with the nearest mean. K-means clustering techniques useful in conjunction with the methods of the invention are known in the art and are described herein.

As used herein, the term “minimization” refers to a process by which the lowest attainable value of a quantity is computed, subject to one or more constraints. The quantity may be, for example, a residual, such as a residual between univariate or multivariate data sets. In the case of residual minimization, the constraints in place may be used to define a relationship between the univariate or multivariate data sets, or to define maximum and/or minimum quantities for one or more values within the data sets.

As used herein, the term “residual” refers to the difference between a known or experimentally determined value and a predicted value for a specified parameter. For instance, when applied to the gene expression profile of a population of cells, a residual refers to the difference between the gene expression profile of a population of cells, for example, as determined empirically (e.g., by the use of a gene or protein expression assay known in the art or described herein, such as PCR, RNA-Seq, or ELISA, among others) and a predicted gene expression profile of the population of cells, for example, as determined from information regarding the gene expression patterns of single cells within the population.

### BRIEF DESCRIPTION OF THE FIGURES

FIG. 1 is a schematic showing the relationship between the gene expression profiles (m) of a collection of multi-cell mixtures (n) (i.e., the “mixture matrix” X_{m×n} shown at the left), the gene expression profiles of a variety of cell types (k) present within the mixtures (i.e., the “signature matrix” S_{m×k} shown in the middle), and the cell type composition of each mixture (i.e., the “concentration matrix” C_{k×n} shown at the right).

FIG. 2 is a graph showing the loge of the ratio of the predicted and actual gene expression signatures per cell type (left y-axis) in a population of 100 multi-cell mixtures as determined by single cell gene profiling. The graph additionally shows the average gene expression signature for each gene and cell type (right y-axis), expressed as 2^{dCT} as determined by qPCR analysis.

FIGS. 3A and 3B are graphs showing the unfiltered and filtered gene expression profiles of single cells sampled from a heterogeneous population of cells. The x and y axes are, respectively, the first and second principal components, as determined by the Principal Component Analysis (PCA) method. Single cell expression profiles were used to determine cell types with common expression patterns by removing cells that show a maximum Pearson correlation to other cells within the sample of below 0.95 and subsequently grouping single cells with common gene expression profiles using K-means clustering. The three large circles depicted in FIG. 3B represent the average gene expression patterns of the three distinct cell types identified by this process.

FIG. 4 is a graph showing the clustering of single cell expression profiles into three like groups using K-means clustering and principle component analysis. The x, y, and z axes are, respectively, the first, second, and third principal components, as determined by the Principal Component Analysis (PCA) method.

FIG. 5 is a schematic showing the process by which full gene expression signatures can be determined based on the cell type composition (i.e., the concentration matrix, C) of various populations of cells. A quadratic program can be used to minimize the residual between the estimated expression of a gene within various mixtures of cell types (the product sC) and the observed signals from expression of the gene of interest (x) in order to determine the full gene expression signature matrix, S.

FIG. 6 is a graph showing the correlation between first and second cell type components in populations of cells generated by simulation and as determined by empirical observation. A total of 92 gene expression profiles were obtained by characterizing the expression of 31 genes by RT-qPCR in single cells isolated from mouse embryos at the 7-8 somite stage. To simulate populations with varying cell type concentrations, cell types were sampled uniformly at random, and the cell type column of the concentration matrix was scaled to a sum of 1. To simulate mixtures, single cells were selected randomly with replacement from each cluster shown in FIG. 4. The gene expression profiles of the chosen cells were summed in order to generate simulated mixtures of cell types.

FIG. 7 is a graph showing the accuracy of inferred mixing proportions of a population of cells for three distinct cell types based on single cell gene expression profiles.

FIG. 8 is a graph showing the concordance of the predicted and actual cell type compositions (i.e., concentration matrices) for three distinct cell types based on single cell gene expression profiles.

FIG. 9 is a graph showing the root mean square error (rmse) of the cell type composition of a population containing three distinct cell types (k=3). The graph plots rmse as a function of the number of genes measured for each of the three cell types, in which genes were added in an arbitrary order.

FIG. 10 is a graph showing the rmse of the cell type composition of a population containing three distinct cell types (k=3). The graph plots rmse as a function of the number of genes measured for each of the three cell types, in which genes were added in the order of their ANOVA F-values.

FIG. 11 is a graph showing the rmse for inferred cell type concentrations and gene expression level of various genes not used for inference for three distinct cell types and simulated populations of 50 cells, as determined by the UCQP deconvolution method (e.g., as described herein). The Foxa2 gene is involved in the regulation of metabolism and in the differentiation of the pancreas and liver. The Nodal gene is involved in mesoderm formation and subsequent organization of axial structures in early embryonic development. The Pax3 is involved in directing fetal development. The Shh gene in involved in modulating patterning of the early embryo. The Wnt3a gene is involved in directing patterning during embryogenesis.

FIG. 12 is a graph showing the rmse for inferred cell type expression profiles and gene expression level of various genes not used for inference for three distinct cell types and simulated populations of 10 cells, as determined by the UCQP deconvolution method (e.g., as described herein).

FIG. 13 is a graph showing the rmse for inferred cell type expression profiles and gene expression level of various genes not used for inference for three distinct cell types and simulated populations of 50 cells, as determined by the UCQP deconvolution method (e.g., as described herein).

FIG. 14 is a violin plot of the true and simulated gene expression distribution of the KIT gene for each of the known cell types. The suffix s and t denotes the simulated and true distributions. The plot demonstrates that the simulated distributions closely resemble the true distributions.

FIGS. 15A-15C compare the accuracy of the four compared deconvolution methods in estimating the concentration matrix using the rmse metric. Each method is evaluated given a different number of mixtures between 5 and 25. In general no method has a strong correlation to the number of mixtures.

FIGS. 16A-16C compare the accuracy of the four compared deconvolution methods in estimating the concentration matrix using the rmse metric. Each method is evaluated given a different error level between mixtures between 5 and 100.

FIGS. 17A-17C compare the accuracy of the four compared deconvolution methods in estimating the concentration matrix using the rmse metric. Each method is evaluated given a different gene count between 8 and 256.

FIG. 18 is a graph having two y-axes. The left y-axis describes the mean absolute error of the signature estimate (solid line), while the right y-axis indicates the rmse of the concentration estimate (dotted line). The number of mixtures is fixed to 5, the error parameter is 100 and the concentrations are random. The x-axis varies the number of mixtures.

FIG. 19 is a graph having two y-axes. The left y-axis describes the mean absolute error of the signature estimate (solid line), while the right y-axis indicates the rmse of the concentration estimate (dotted line). The number of mixtures is fixed to 20, the gene count is 64, concentrations are random. The x-axis varies the error parameter.

### DETAILED DESCRIPTION

Cell or tissue type heterogeneity is present in data collected from numerous biological sources. It is typically difficult or impossible to physically separate cell types in any given mixture. Computational gene expression deconvolution is the process by which this separation is done in silico. One application of deconvolution is assisting stem cell biologists to obtain whole-transcriptome expression profiles of closely related cell types. Described herein are methods of determining gene expression profiles of related cell types using single-cell gene profiling (e.g., by quantitative PCR (qPCR) or deep sequencing techniques, such as RNA-Seq) of a small number of genes to aid in the deconvolution of whole-transcriptome profiles of mixed samples.

The expression profiles of m genes measured in n mixtures of k cell types are modeled as X=SC, where X is a m×n matrix whose columns are the expression profiles of individual mixtures, S is m×k signature matrix whose columns are expression profiles of individual cell types, and C is a k×n concentration matrix whose columns represent the proportions of each cell type in individual mixtures. There are a variety of approaches used to solve the deconvolution problem and they can be classified based on the expected input and output.

One formulation frames the problem as estimating S when the mixtures X and concentrations C are known. Typically, the concentrations are measured by some external technique such as fluorescence activated cell sorting (FACS) or fluorescent in situ hybridization (FISH). Another formulation is to assume X and S are known, and the goal is to estimate C. This formulation is often used when studying well-characterized cell types, such as cells of the hematopoietic lineage, and is often referred to as supervised deconvolution. Unsupervised deconvolution is the simultaneous estimation of both S and C given X. This scenario is much more challenging. Finally, the semi-supervised formulation assumes that existing information such as unique marker genes are available to describe each cell type.

The present invention is based on a novel variation of these previously proposed themes utilizing single-cell expression profiles denoted as Z=z1, z2, . . . zq, where q is the number of single cells available, in addition to bulk mixture gene expression data. Typically these data are generated using a microfluidic device that performs qPCR or RNA-Seq reactions on each single-cell. These additional data can be pre-processed into the canonical cell type signatures using supervised deconvolution approaches described herein.

Thus, the invention provides the means to deconvolve population level (i.e. mixtures) given a small number of single-cell resolution measurements from the same sample. By m herein will be denoted the number of genes, by k the number of cell types, and by n the number of samples.

The gene expression level measured by PCR (e.g., qPCR) for a gene i is typically given as the threshold cycle Cⁱ_{T}. This measurement varies logarithmically with the abundance level.

The Cⁱ_{T} values are often normalized using a constantly expressed housekeeping gene (or the geometric average of several housekeeping genes) in order to account for variances in starting material which would effect detection threshold. Thus the normalized values are reported as

ΔCⁱ_{T}=Cⁱ_{Ttarget}−Cⁱ_{Treferencegene}

The ΔCⁱ_{T} values can be converted to a linear scale by using

2^{-ΔC}^{ⁱ}^{_{T}}

as expression level.

A mixture is a heterogeneous collection of cells where the expression levels of m selected genes are measured using, e.g., qPCR. A single measurement is denoted by the vector x, where |x|=m. A set of n mixtures is denoted as the mixture matrix X with dimensions m×n.

The signature for a given cell type is denoted as a vector s, where |s|=m, and each element in the vector is the mean expression value of each gene in cells of this type. The complete set of signatures for all cell types is denoted as S where S is an m×k matrix.

Each mixture is assumed to be a linear combination of cell type signatures. For one mixture the concentration of each cell type is denoted by a vector c, where |c|=k. A set of n concentration vectors is denoted as the concentration matrix C with dimensions k×n.

Thus, expression data of a set of mixtures is modeled as

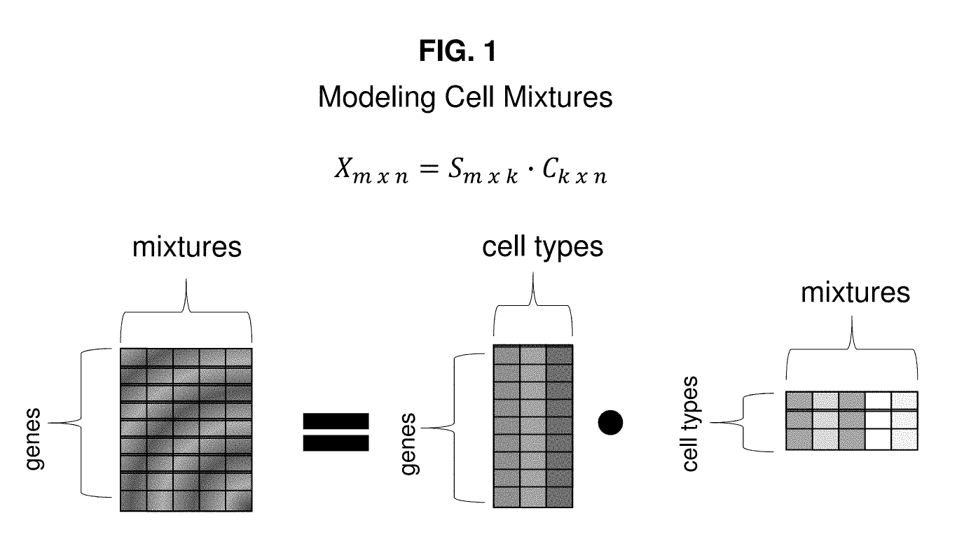

X=SC

where each heterogeneous mixture is a linear combination of cell type signatures with concentrations specified by the columns of C, as shown, for example, in FIG. 1.

### Determining Gene Expression of a Missing Cell Type by Residual Minimization

With the knowledge of the gene expression profile of a population of cells (e.g., a heterogeneous population of cells) and the gene expression profile of a subset of cell types present within the population, one can calculate the gene expression profile of a rare cell type present within the population of cells using residual minimization. Described herein is a two-step approach whereby single cells are first clustered into representative cell types and the signature matrix is computed. Next, the mixing proportions matrix C is estimated using quadratic programming. This enables the determination of the gene expression profile of the full set of cell types present within the population, including that of a rare or difficult-to-detect cell type that may not have been sampled at the single cell level due to low abundance. The methods described herein are useful, e.g., for determining the gene expression pattern of biologically significant cell types (such as progenitor cells, pluripotent stem cells, and/or diseased cells) that are often intractable to characterization by single cell profiling.

### Signature Generation

One of the core challenges of single cell genetics is the meaningful classification or grouping of cells. For the purposes of the methodology described herein, the term cell type will be broadly defined as a cell phenotype that is statistically separable based on gene expression data. The signature matrix S_{m×k} is built by clustering single-cell gene expression data (e.g., qPCR data) z1, z2, . . . into k clusters. This represents an instance of unsupervised learning, where samples need to be labeled based on their gene expression vectors. Various strategies can be used for the clustering of gene expression data into like groups, for example, by minimizing the distance between samples in a cluster or by grouping functionally related samples. K-means clustering is another paradigm for grouping the single-cell data and offers the advantage of explicitly allowing the investigator to control the number of theoretical cell types. The average expression profile of each single-cell in a cluster can be used to create the cell type signature matrix S_{m×k}.

### Concentration Matrix

The next task is to solve for the concentration matrix C_{k×n}. This can be done utilizing methodology described, e.g., in Gong et al., PLoS One 6:e27156, 2011, the disclosure of which is incorporated herein by reference in its entirety. Each column of X corresponds to genes measured, e.g., by a gene expression detection technique described herein, such as qPCR, and is a linear combination of single cell expression profiles with unknown concentrations. A particular column in X can be denoted as x and its corresponding column in C by c. Inferring c can be formulated as the following quadratic program:

$$\begin{matrix}minimize & {‖ Sc - x ‖}_{2} \\ subject to & \sum c = 1 \\ & {c}_{i} \geq 0 ∀ i = 0 … m\end{matrix}$$

This least-squares formulation can be solved with any constrained quadratic programming solver.

### Characterizing the Gene Expression Profile of a Missing Cell Type

Solving for a missing cell type is an extension of the above computational deconvolution framework. The signature matrix including the missing cell type will be denoted as:

Ŝ_{m×(k+1)}

and the concentration matrix will be denoted as:

\hat{C}(k+1)×n:

The goal is to simultaneously calculate the missing k^{th} column of signature matrix and the complete concentration matrix. The quadratic formulation is the following:

$$\begin{matrix}minimize & {‖ \hat{S} \hat{C} - X ‖}_{2} \\ subject to & \sum c = 1 \\ & {c}_{i} \geq 0 ∀ i = 0 … m \\ & {s}_{k} \geq 0\end{matrix}$$

This is a non-linear non-negative least squares optimization problem. Damped least-squares (DLS) is used here to solve the optimization problem. The missing cell type signature is initialized as the average of all the known cell types and the concentrations are set uniformly.

One additional challenge is determining if there is a missing cell type present in the mixtures. This is addressed by comparing the residual value of the system with:

\hat{r}=|X−ŜĈ|

and without:

r=|X−SC|

the missing cell type, respectively.

If \hat{r}>r+γ where γ is some small constant then our approach will report that a missing cell type is likely present. The small constant is in place in order to require a non-trivial difference between the two scenarios.

### Methods of Measuring Gene Expression

The expression level of a gene expressed by a single cell, type of cell, or plurality of cells can be ascertained, for example, by evaluating the concentration or relative abundance of RNA transcripts (e.g., mRNA, small RNA, siRNA, miRNA, rRNA, tRNA, or snRNA) derived from transcription of a gene of interest, or a DNA equivalent thereof (e.g., cDNA). Additionally or alternatively, gene expression can be determined by evaluating the concentration or relative abundance of protein produced by transcription and translation of a gene of interest. Protein concentrations can also be assessed using functional assays, such as enzymatic assays or gene transcription assays in the event the gene of interest encodes an enzyme or a modulator of transcription, respectively.

Methods for measuring the levels of RNAs, DNAs, and/or proteins are well known in the art. The sections that follow describe exemplary techniques that can be used to measure the expression level of one or more genes, for instance, at the level of a single cell or a population of cells. Expression of genes in a sample can be analyzed by a number of methodologies, many of which are known in the art and understood by the skilled artisan, including, but not limited to, nucleic acid sequencing, microarray analysis, proteomics, in situ hybridization (e.g., fluorescence in-situ hybridization (FISH)), amplification-based assays, fluorescence activated cell sorting (FACS), northern analysis and/or PCR analysis of mRNAs (or DNA equivalents thereof).

### Nucleic Acid Detection

Nucleic acid-based datasets suitable for analysis in conjunction with the methods of the invention include gene expression profiles. Such profiles may include whole transcriptome sequencing data (e.g., RNA-Seq data), panels of mRNAs, noncoding RNAs, or any other nucleic acid sequence that may be expressed from genomic DNA. Other nucleic acid datasets suitable for use with the methods of the invention may include expression data collected by imaging-based techniques (e.g., Northern blotting or Southern blotting). Northern blot analysis is a conventional technique well known in the art and is described, for example, in Molecular Cloning, a Laboratory Manual, second edition, 1989, Sambrook, Fritch, Maniatis, Cold Spring Harbor Press, 10 Skyline Drive, Plainview, N.Y. 11803-2500. Typical protocols for evaluating the status of genes and gene products are found, for example in Ausubel et al., eds., 1995, Current Protocols In Molecular Biology, Units 2 (Northern Blotting), 4 (Southern Blotting), 15 (Immunoblotting) and 18 (PCR Analysis).

Gene expression profiles to be analyzed in conjunction with the methods described herein may include, for example, microarray data or nucleic acid sequencing data produced by a sequencing method known in the art (e.g., Sanger sequencing and next-generation sequencing methods, also known as high-throughput sequencing or deep sequencing). Exemplary next generation sequencing technologies include, without limitation, Illumina sequencing, Ion Torrent sequencing, 454 sequencing, SOLiD sequencing, and nanopore sequencing platforms. Additional methods of sequencing known in the art can also be used. For instance, mRNA expression levels may be determined using RNA-Seq (e.g., as described in Mortazavi et al., Nat. Methods 5:621-628, 2008, the disclosure of which is incorporated herein by reference in their entirety). RNA-Seq is a robust technology for monitoring expression by direct sequencing the RNA molecules in a sample. Briefly, this methodology may involve fragmentation of RNA to an average length of 200 nucleotides, conversion to cDNA by random priming, and synthesis of double-stranded cDNA (e.g., using the Just cDNA DoubleStranded cDNA Synthesis Kit from Agilent Technology). Then, the cDNA is converted into a molecular library for sequencing by addition of sequence adapters for each library (e.g., from Illumina®/Solexa), and the resulting 50-100 nucleotide reads are mapped onto the genome.

Gene expression levels may be determined using microarray-based platforms (e.g., single-nucleotide polymorphism (SNP) arrays), as microarray technology offers high resolution. Details of various microarray methods can be found in the literature. See, for example, U.S. Pat. No. 6,232,068 and Pollack et al., Nat. Genet. 23:41-46, 1999, the disclosures of each of which are incorporated herein by reference in their entirety. Using nucleic acid microarrays, mRNA samples are reverse transcribed and labeled to generate cDNA. The probes can then hybridize to one or more complementary nucleic acids arrayed and immobilized on a solid support. The array can be configured, for example, such that the sequence and position of each member of the array is known. Hybridization of a labeled probe with a particular array member indicates that the sample from which the probe was derived expresses that gene. Expression level may be quantified according to the amount of signal detected from hybridized probe-sample complexes. A typical microarray experiment involves the following steps: 1) preparation of fluorescently labeled target from RNA isolated from the sample, 2) hybridization of the labeled target to the microarray, 3) washing, staining, and scanning of the array, 4) analysis of the scanned image and 5) generation of gene expression profiles. One example of a microarray processor is the Affymetrix GENECHIP® system, which is commercially available and comprises arrays fabricated by direct synthesis of oligonucleotides on a glass surface. Other systems may be used as known to one skilled in the art.

Amplification-based assays also can be used to measure the expression level of one or more markers (e.g., genes). In such assays, the nucleic acid sequences of the gene act as a template in an amplification reaction (for example, PCR, such as qPCR). In a quantitative amplification, the amount of amplification product is proportional to the amount of template in the original sample. Comparison to appropriate controls provides a measure of the expression level of the gene, corresponding to the specific probe used, according to the principles described herein. Methods of real-time qPCR using TaqMan probes are well known in the art. Detailed protocols for real-time qPCR are provided, for example, in Gibson et al., Genome Res. 6:995-1001, 1996, and in Heid et al., Genome Res. 6:986-994, 1996, the disclosures of each of which are incorporated herein by reference in their entirety. Levels of gene expression as described herein can be determined by RT-PCR technology. Probes used for PCR may be labeled with a detectable marker, such as, for example, a radioisotope, fluorescent compound, bioluminescent compound, a chemiluminescent compound, metal chelator, or enzyme.

### Protein Detection

Gene expression can additionally be determined by measuring the concentration or relative abundance of a corresponding protein product encoded by a gene of interest. Protein levels can be assessed using standard detection techniques known in the art. Examples of protein expression analysis that generate data suitable for use with the methods described herein include, without limitation, proteomics approaches, immunohistochemical and/or western blot analysis, immunoprecipitation, molecular binding assays, ELISA, enzyme-linked immunofiltration assay (ELIFA), mass spectrometry, mass spectrometric immunoassay, and biochemical enzymatic activity assays. In particular, proteomics methods can be used to generate large-scale protein expression datasets in multiplex. Proteomics methods may utilize mass spectrometry to detect and quantify polypeptides (e.g., proteins) and/or peptide microarrays utilizing capture reagents (e.g., antibodies) specific to a panel of target proteins to identify and measure expression levels of proteins expressed in a sample (e.g., a single cell sample or a multi-cell population).

Exemplary peptide microarrays have a substrate-bound plurality of polypeptides, the binding of an oligonucleotide, a peptide, or a protein to each of the plurality of bound polypeptides being separately detectable. Alternatively, the peptide microarray may include a plurality of binders, including but not limited to monoclonal antibodies, polyclonal antibodies, phage display binders, yeast two-hybrid binders, aptamers, which can specifically detect the binding of specific oligonucleotides, peptides, or proteins. Examples of peptide arrays may be found in U.S. Pat. Nos. 6,268,210, 5,766,960, and 5,143,854, the disclosures of each of which are incorporated herein by reference in their entirety.

Mass spectrometry (MS) may be used in conjunction with the methods described herein to identify and characterize the gene expression profile of a single cell or multi-cell population. Any method of MS known in the art may be used to determine, detect, and/or measure a peptide or peptides of interest, e.g., LC-MS, ESI-MS, ESI-MS/MS, MALDI-TOF-MS, MALDI-TOF/TOF-MS, tandem MS, and the like. Mass spectrometers generally contain an ion source and optics, mass analyzer, and data processing electronics. Mass analyzers include scanning and ion-beam mass spectrometers, such as time-of-flight (TOF) and quadruple (Q), and trapping mass spectrometers, such as ion trap (IT), Orbitrap, and Fourier transform ion cyclotron resonance (FT-ICR), may be used in the methods described herein. Details of various MS methods can be found in the literature. See, for example, Yates et al., Annu. Rev. Biomed. Eng. 11:49-79, 2009, the disclosure of which is incorporated herein by reference in its entirety.

Prior to MS analysis, proteins in a sample can be first digested into smaller peptides by chemical (e.g., via cyanogen bromide cleavage) or enzymatic (e.g., trypsin) digestion. Complex peptide samples also benefit from the use of front-end separation techniques, e.g., 2D-PAGE, HPLC, RPLC, and affinity chromatography. The digested, and optionally separated, sample is then ionized using an ion source to create charged molecules for further analysis. Ionization of the sample may be performed, e.g., by electrospray ionization (ESI), atmospheric pressure chemical ionization (APCI), photoionization, electron ionization, fast atom bombardment (FAB)/liquid secondary ionization (LSIMS), matrix assisted laser desorption/ionization (MALDI), field ionization, field desorption, thermospray/plasmaspray ionization, and particle beam ionization. Additional information relating to the choice of ionization method is known to those of skill in the art.

After ionization, digested peptides may then be fragmented to generate signature MS/MS spectra. Tandem MS, also known as MS/MS, may be particularly useful for methods described herein allowing for ionization followed by fragmentation of a complex peptide sample, such as a sample obtained from a multi-cell population described herein. Tandem MS involves multiple steps of MS selection, with some form of ion fragmentation occurring in between the stages, which may be accomplished with individual mass spectrometer elements separated in space or using a single mass spectrometer with the MS steps separated in time. In spatially separated tandem MS, the elements are physically separated and distinct, with a physical connection between the elements to maintain high vacuum. In temporally separated tandem MS, separation is accomplished with ions trapped in the same place, with multiple separation steps taking place over time. Signature MS/MS spectra may then be compared against a peptide sequence database (e.g., SEQUEST). Post-translational modifications to peptides may also be determined, for example, by searching spectra against a database while allowing for specific peptide modifications.

### Applications

The methods described herein may be used, for example, for the diagnosis and/or monitoring of disease states. For instance, the gene profiling methods described herein can be used to determine the gene expression patterns of, for instance, cancer cells, autoimmune cells, aging cells, or other cells involved in the propagation, initiation, and/or progression, of any disease known in the art. Such cells may be rare cell types (i.e., cell types present at low abundance in a sample as described herein). As such, the methods of the invention may be useful for determining the expression profile of such disease cells. The methods described herein can be applied to diagnosis, for instance, by determining the gene expression profile of a cell type of interest (e.g., a cancer cell, autoimmune cell, or aging cell) and/or detecting the presence of the cell in a sample isolated from a subject (e.g., a human subject) based on the gene expression profile of the target cell type. The methods described herein can additionally be applied to monitoring the progression of a disease, as one of skill in the art may determine the gene expression profile of a cell type of interest (e.g., a cancer cell, autoimmune cell, or aging cell) and detect the presence of the target cell type in a sample isolated from a subject (e.g., a human subject) one or more times in order to monitor the progression of the disease.

In one example, a physician of skill in the art may withdraw a sample from a human subject (e.g., a blood sample) and use the methods described herein to determine the gene expression profile of a cancerous cell or an autoreactive cell, such as an autoreactive T-cell. If it is determined that, e.g., one or more cancer cells or autoreactive T-cells are present, this finding is indicative that the patient may have a cancerous or autoimmune pathology, respectively.

The methods described herein can additionally be used to monitor the progression of a disease. In one example, a physician of skill in the art may withdraw a sample from a subject (e.g., a human subject) one or more times (e.g., over the course of 1 week, 2 weeks, 3 weeks, 4 weeks, 5 weeks, 6 weeks, 7 weeks, 8 weeks, 9 weeks, 10 weeks, 15 weeks, 20 weeks, 25 weeks, 30 weeks, 35 weeks, 40 weeks, 45 weeks, 50 weeks, 1 year, 2 years, 3 years, 4 years, 5 years, or more) in order to ascertain, for example, whether disease cells (e.g., cancerous or autoreactive cells) previously identified remain present within the subject, or whether such disease cells may be newly appeared in the subject. These findings can provide insight into the initiation, progression, and/or propagation of the disease.

### EXAMPLES

The following examples are put forth so as to provide those of ordinary skill in the art with a description of how the methods described herein may be used and evaluated, and are intended to be purely exemplary of the invention and are not intended to limit the scope of what the inventors regards as their invention.

### Example 1

### Whole Transcriptome Deconvolution Using Single-Cell Data

The invention features, in one example, a method that utilizes single-cell qPCR probing of a small number of genes to aid in the deconvolution of whole-transcriptome profiles of mixed samples. In this method, the expression profiles of m genes measured in n mixtures of k cell types are modelled as X=SC, where X is a m×n matrix whose columns are the expression profiles of individual mixtures, S is a m×k signature matrix whose columns are expression profiles of individual cell types, and C is a k×n concentration matrix whose columns represent the proportions of each cell type in individual mixtures. In this abstract we will assume that individual cell types as well as a reduced signature matrix S can be reliably inferred from single-cell qPCR data generated for a small subset of genes.

### Methods

### A. Constructing Reduced Cell-Type Signatures

1) Noise Reduction. Due to large biological and technical noise in single-cell qPCR data, we applied a technique in which each sample was required to have 0.95 Pearson correlation with at least one other sample, otherwise it is removed.

2) K-means Clustering. We chose to use k-means clustering to group the gene expression data from single-cell data because it explicitly allows us to control the number of theoretical cell types. The average expression profile of each single cell in a cluster is used to create the reduced cell type signature matrix Ŝ.

### B. Estimate Mixing Proportions

The next task is to solve for the concentration matrix C. We compute the concentration matrix describing the mixtures using methods well known in the art. Each column of X, and hence also columns of the reduced expression matrix \hat{X}, obtained by retaining only rows of X corresponding to genes measured by qPCR, is a linear combination of single cell expression profiles with unknown concentrations. Let us denote that a particular column in \hat{X} as x and its corresponding column in C by c. Inferring c can be formulated as the following quadratic program that can be solved using standard constrained quadratic programming solvers:

$$\begin{matrix}minimize & {‖ \hat{S} c - x ‖}_{2} \\ subject to & \sum c = 1 \\ & {c}_{i} \geq 0 ∀ i = 0 … m\end{matrix}$$

### C. Estimate Full Expression Signatures

It is still necessary to estimate the signatures of the full gene profile. Using the concentration matrix C inferred in the previous step, the gene signature s of a gene not measured by qPCR can be inferred from the mixed gene expression data x using a similar least squares quadratic program:

$$\begin{matrix}minimize & {‖ \hat{S} c - x ‖}_{2} \\ subject to & \sum c = 1 \\ & {c}_{i} \geq 0 ∀ i = 0 … m\end{matrix}$$

### Preliminary Experimental Results

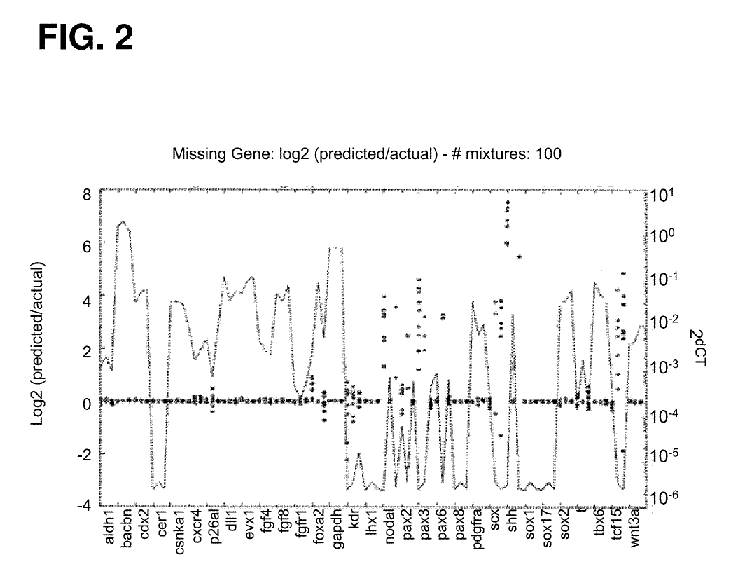

The above method was applied on qPCR expression data generated from mouse embryos at the 7-8 somite stage. Expression levels of 31 genes were characterized by RT-qPCR for 97 single cells and 12 mixed samples. In order to test the method's ability to estimate the concentration matrix and complete the gene signature, we ran a leave-one-out experiment on each gene. FIG. 2 demonstrates that the method is able to accurately deconvolve expression levels of most genes.

### Example 2

### Determining the Gene Expression Profile of a Low-Abundance Progenitor Cell

In some instances, the methods described herein may be used to determine whole transcriptome expression profiles of individual cell types, e.g., despite the technical challenge of measuring gene expression in rare and low-abundance cell types at the single cell level. The likelihood that such cells will be sampled in single cell experiments is proportional to the frequency with which these cell types appear in the population from which the sample is isolated. The gene expression profiles of such cells (e.g., progenitor cells or pluripotent stem cells) may provide valuable insight as to those genes involved in the differentiation of cells in a particular lineage and/or those that dictate various important developmental events.

To circumvent the limitations associated with sampling rare and low-abundant cells, the methods described herein can be used to approximate the gene expression profile of cell types present within a population of cells by computational deconvolution of multi-cell mixtures. This approach relies on the use of single cell gene expression data (e.g., qPCR data or deep sequencing data) obtained for a small quantity of genes. This single-cell assisted deconvolution technique represents an instance in which the matrix vector, X, of gene expression data of an entire population of cells and of individual cells is provided or experimentally determined, e.g., by qPCR. From this information, the signature matrix, S, of genes expressed per each cell type present within the population, and the concentration matrix, C, of the quantity and identity of cell types present within the population can be inferred.

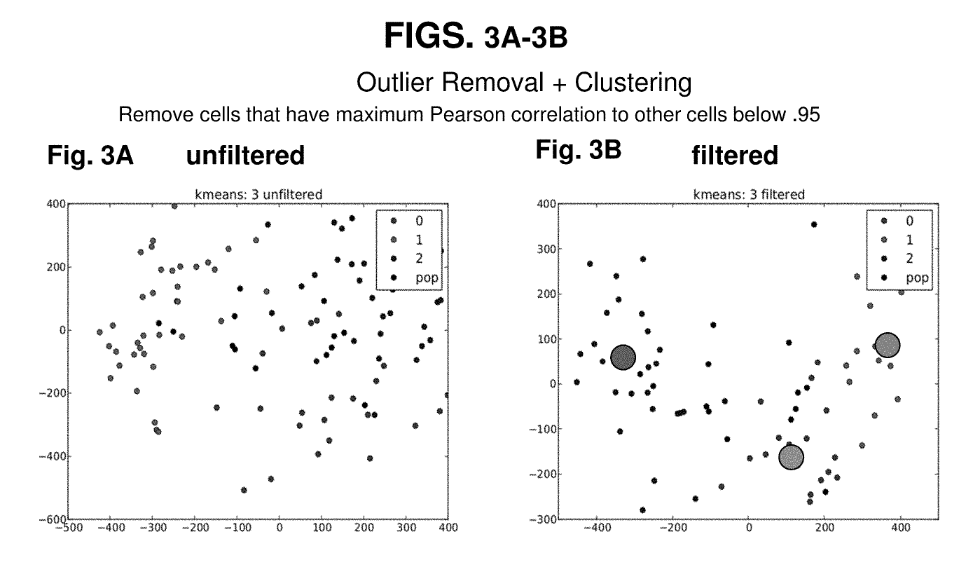

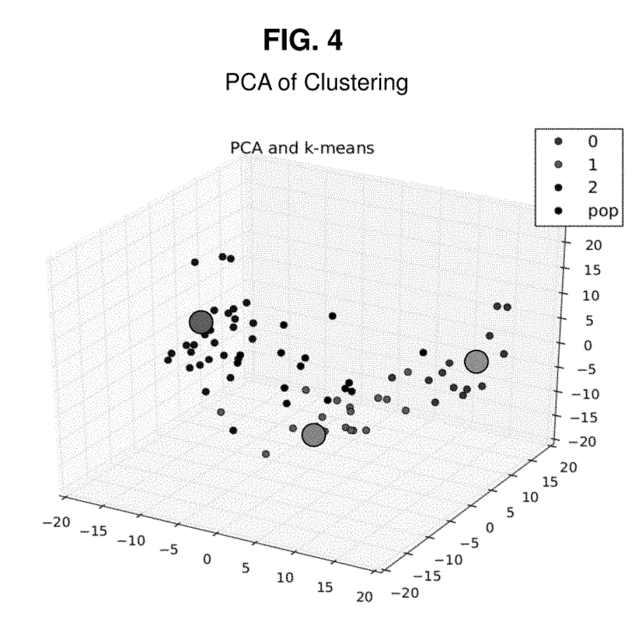

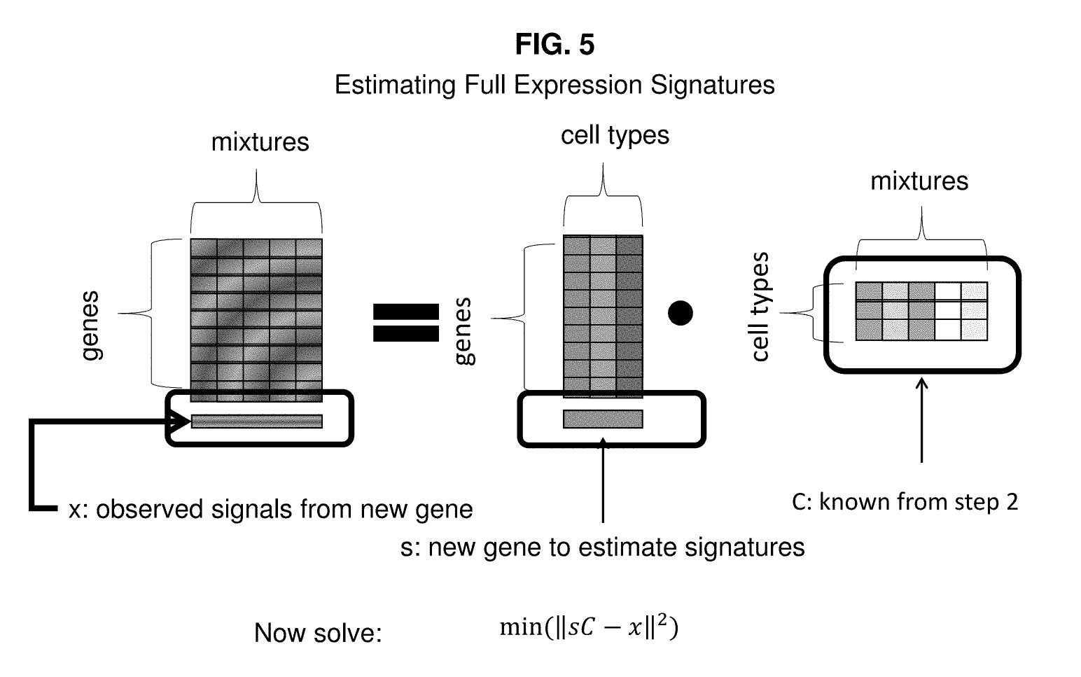

The overarching steps of this approach include identifying cell types and estimating a reduced signature matrix, Ŝ, using single-cell gene expression data. This process involves transforming the single-cell gene profiles into clusters of common cell types by correlating single-cell expression profiles, removing outliers present within the dataset, and performing clustering (e.g., K-means clustering) and averaging of the gene expression data to determine a set of cell types that share common gene expression patterns. These correlating and clustering processes are represented in FIGS. 3 and 4. The reduced signature matrix, Ŝ, can then be used to determine the concentration matrix, C, of the quantity and identity of cell type present within the population using a quadratic program, one mixture at a time. The full gene expression signature matrix, S, can then be determined using the concentration matrix, C, using a similar quadratic program, one gene at a time, as shown in FIG. 5.

For instance, the concentration matrix for a given mixture, i, can be determined according to the formula:

$$min ( {‖ \hat{S} c - x ‖}^{2} ) , s . t . \{ \begin{matrix}\sum c = 1 \\ {c}_{l} \geq 0 ∀ l = 0 … k\end{matrix} \\ x = {X}_{j , i} ∀ j = 1 … m \\ c = {C}_{l , i} ∀ l = 1 … k$$

wherein S denotes the reduced signature matrix as determined based on the centroid values of the K-means clusters, e.g., shown in FIG. 3.

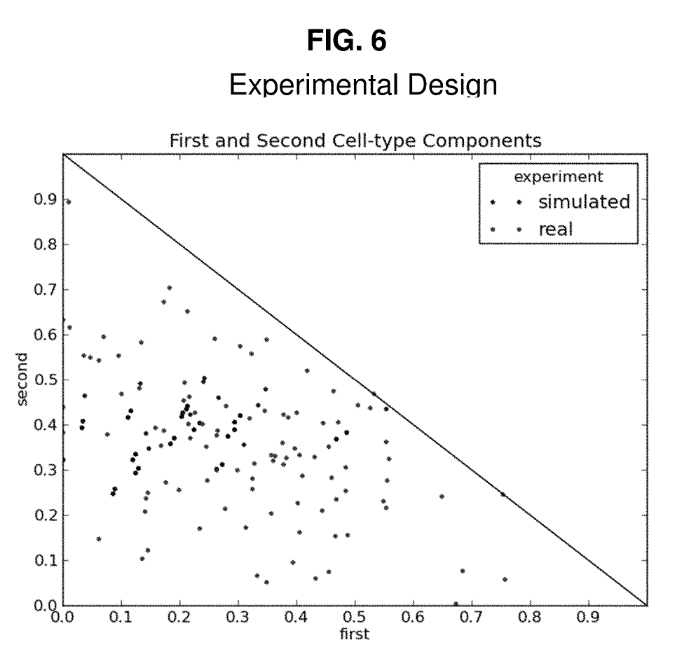

The design of an exemplary application of this procedure is summarized in FIG. 6. Single cell expression profiles of 92 individual cells across 31 unique genes were determined by qPCR. The data were analyzed by determining C_{T} values for each gene that correspond to the PCR cycle in which the gene was detected. Gene expression was normalized relative to the geometric mean expression level of house-keeping genes (GAPDH and BACTIN1). The relative gene expression level of a gene, x, denoted dCT(x), was determined by the formula:

dCT(x)=geometric mean−CT(x)

The corresponding expression level was determined according to the formula:

expression(x)=2̂dCT(x)

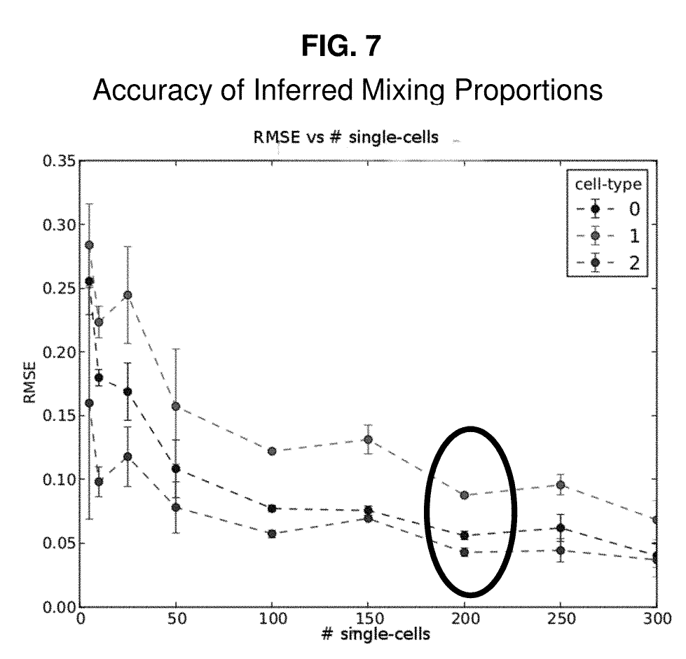

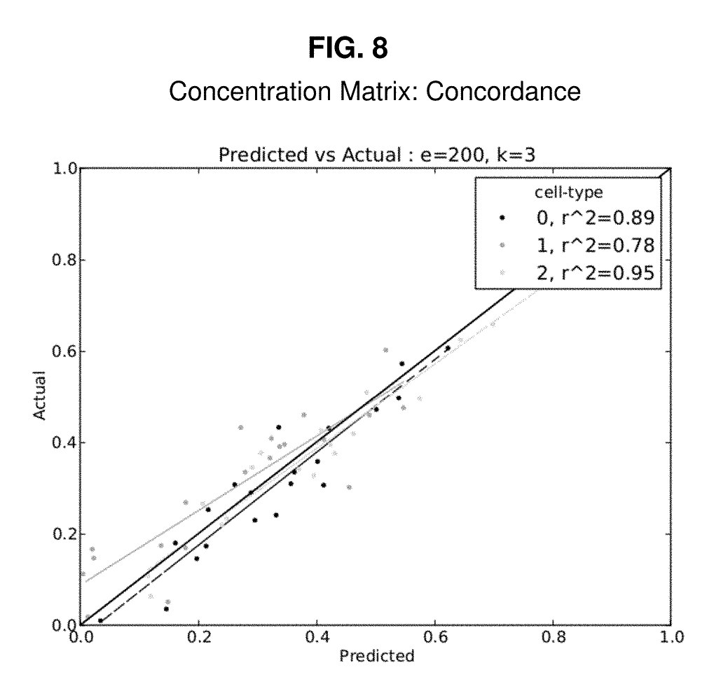

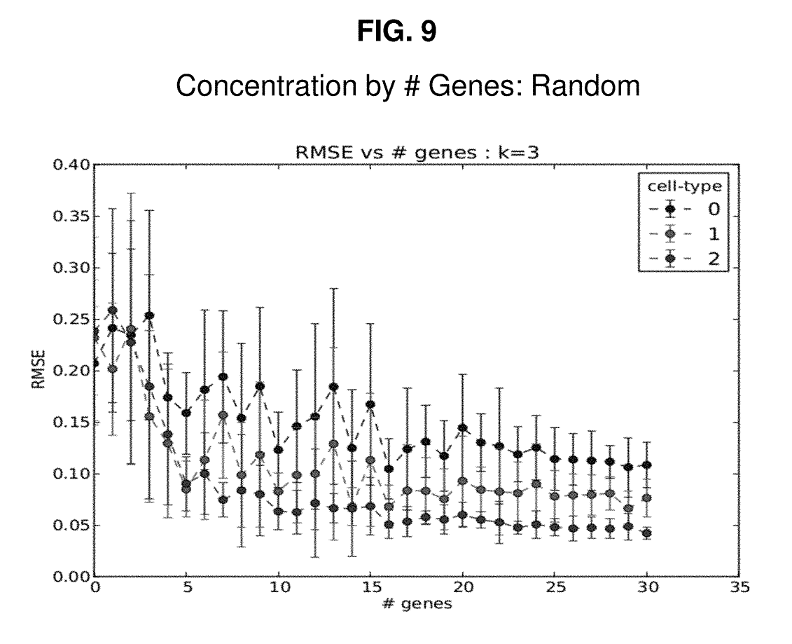

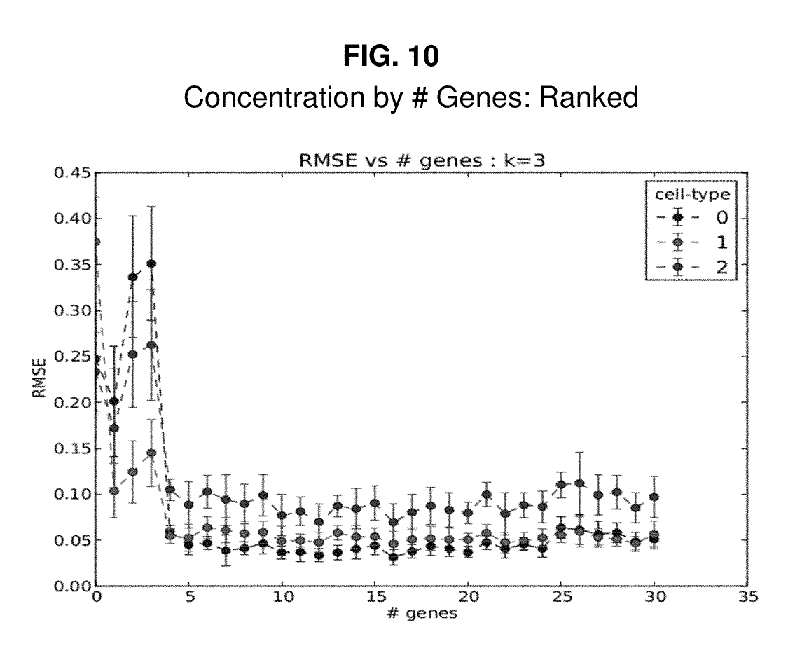

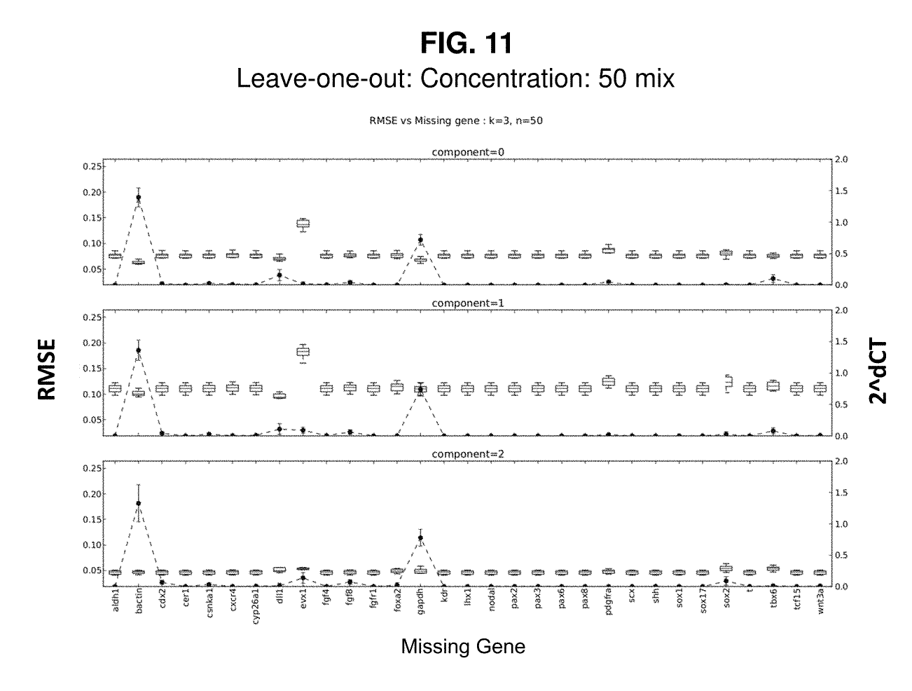

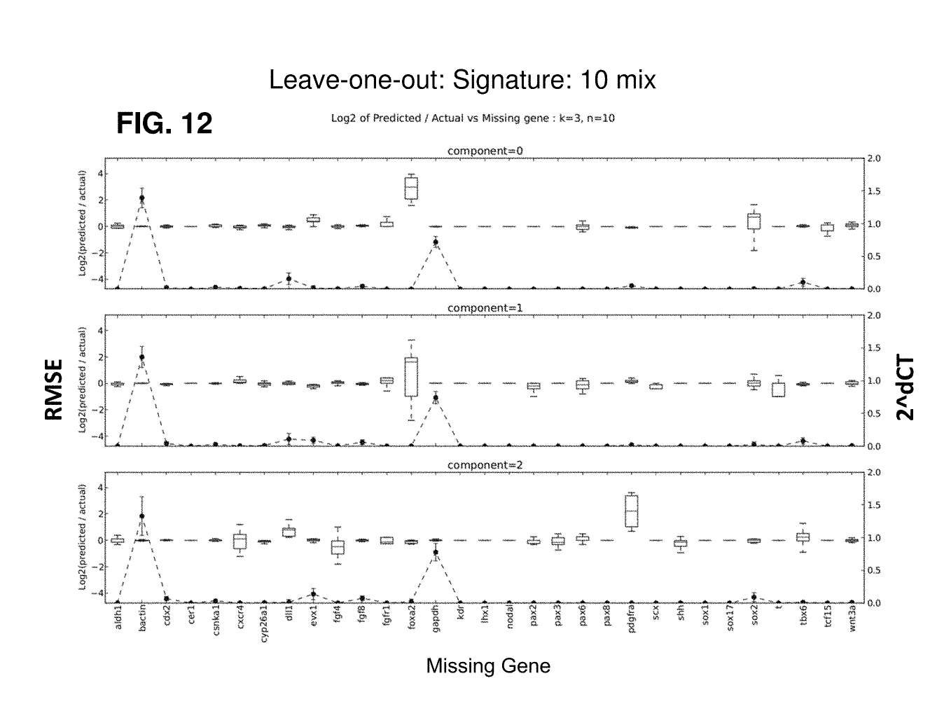

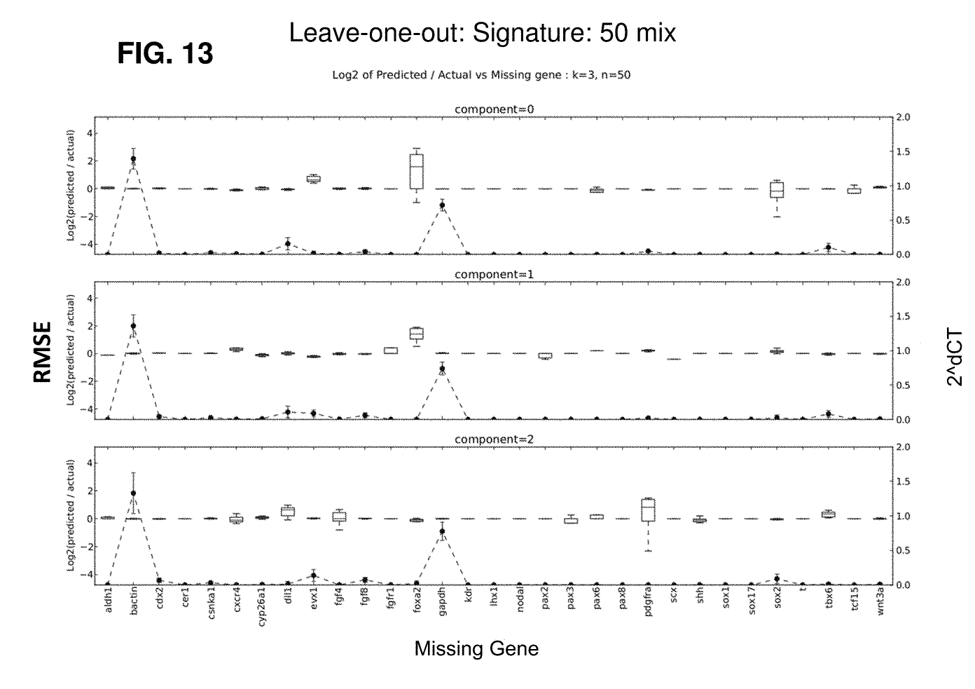

A simulated concentration matrix was determined by sampling uniformly at random and scaling the cell type composition column such that the sum of this column is 1. In order to simulate mixtures, single cells were chosen randomly with replacement from each cluster. The gene expression profiles of these cells were summed to generate a simulated mixture. The matrices determined using the above methods are described in FIGS. 7-13.

The methods described herein can be extrapolated to report a confidence interval of estimate concentration and signature matrices. In these cases, large confidence intervals would be expected to correlate with poor accuracy. Additionally, heterogeneous gene profiling techniques can be used to obtain gene expression data for use with the methods described herein. For instance, qPCR can be used to obtain single cell expression profiles, and RNA-Seq can be used to obtain gene expression profiles for multi-cell mixtures. These data can subsequently be normalized and used directly in the programs described above. The techniques described herein can also be applied to whole-genome scale investigations, e.g., so as to estimate about 5, 10, 50, 100, 1,000, 10,000, or more gene signature matrices.

### Example 3

### Single-Cell Assisted Deconvolution

In one example, a simple existing deconvolution method is enhanced to yield greater accuracy than methods which do not take advantage of single cell data.

### Motivation

Cell or tissue type heterogeneity is present in data collected from numerous biological sources. It is generally difficult or impossible to physically separate cell types in any given mixture. Computational gene expression deconvolution is the process by which this separation is done in silico. In some instances, such deconvolution may be used to obtain whole-transcriptome expression profiles of closely related cell types (e.g., stem cells). In this example, single-cell qPCR probing of a small number of genes is used to aid in the deconvolution of whole-transcriptome profiles of mixed samples.

The expression profiles of m genes measured in n mixtures of k cell types are modeled as

X=SC,

in which X is a m×n matrix whose columns are the expression profiles of individual mixtures, S is m×k signature matrix whose columns are expression profiles of individual cell types, and C is a k×n concentration matrix whose columns represent the proportions of each cell type in individual mixtures. There are a variety of approaches used to solve the deconvolution problem and they can be classified based on the expected input and output.

One formulation used by propose the problem as estimating S when the mixtures X and concentrations C are known. The concentrations can be measured by any technique known in the art, for example, fluorescence-activated cell sort (FACS) or fluorescence in situ hybridization (FISH). Another formulation is to assume X and S are known, and the goal is to estimate C. This formulation is often found when studying extensively studied cell types such as the blood lineage and is often referred to as supervised deconvolution. Unsupervised deconvolution is the simultaneous estimation of both S and C given X. This scenario is much more challenging, however several approaches have yielded promising results. Finally the semi-supervised formulation assumes that existing information such as unique marker genes are available to describe each cell type.

This work proposes a novel variation on these previously proposed themes where in addition to the bulk mixture data, single-cell expression profiles denoted as Z=Z1, Z2, . . . q where q is the number of single cells available. Typically this comes from a microfluidic device that performs qPCR or RNA-Seq reactions on each single-cell. This additional data can be pre-processed into the canonical cell type signatures where typical supervised deconvolution approaches are applicable.

One common application of single cell genetics is the detection of rare cell types. These rare cells can be progenitor stem cells which will only ever be present at an extremely low abundance, or potentially the cell type is rare because it is a subtle response to changes in environmental conditions. The likelihood that these rare cells are sample in the single cell experiments is proportional to its frequency. This makes experimentally measuring extremely rare cells quite difficult. This work will present first steps towards computationally inferring the canonical signature of rare cell types which cannot be directly measured.

### Problem Definition

In this example, a method is described for deconvolving population level (e.g., mixtures) given a small number of single-cell resolution measurements from the same sample. We will denote by m the number of genes, by k the number of cell types, and by n the number of samples.

The gene expression level measured by qPCR for a gene i is generally given as the threshold cycle Cⁱ_{T}. This measurement varies logarithmically with the abundance level.

The Cⁱ_{T} values may be normalized using a constantly expressed housekeeping gene (or the geometric average of several housekeeping genes) in order to account for variances in starting material which would effect detection threshold. Thus the normalized values are reported as

ΔCⁱ_{T}=Cⁱ_{Ttarget}−Cⁱ_{Treferencegene}

The ΔCⁱ_{T} values can be converted to a linear scale by using

2^{-ΔC}^{ⁱ}^{_{T}}

as expression level.

A mixture is generally a heterogeneous collection of cells where the expression levels of m selected genes are measured using qPCR. A single measurement is denoted by the vector x, where |x|=m. A set of n mixtures is denoted as the mixture matrix X with dimensions m×n.

The signature for a given cell type is denoted as a vector s, where |s|=m, an each element in the vector is the mean expression value of each gene in cells of this type. The complete set of signatures for all cell types is denoted as S where S is an m×k matrix.

Each mixture is assumed to be a linear combination of cell-type signatures. For one mixture the concentration of each cell type is denoted by a vector C, where |c|=k. A set of n concentration vectors is denoted as the concentration matrix C with dimensions k×n.

Thus, expression data of a set of mixtures is modeled as

X=SC

where each heterogeneous mixture is a linear combination of cell type signatures with concentrations specified by the columns of C.

### Method

We use a two-step approach whereby single cells are first clustered into representative cell types and the signature matrix is computed. Next the mixing proportions matrix C is estimated using quadratic programming.

Signature Generation

One of the core challenges of single cell genetics is the meaningful classification or grouping of cells. For the purposes of this work the term cell type will be broadly defined as a cell phenotype that is statistically separable based on gene expression data. The signature matrix S_{m×k} is built by clustering the single-cell qPCR data Z1, Z2, . . . Zq into k clusters. This problem is an instance of unsupervised learning, where samples need to be labeled based on their gene expression vectors. Numerous objectives have been proposed such as minimizing the distance between samples in a cluster, and others focus on grouping functionally related samples. K-means clustering was chosen to group the single-cell data because it explicitly allows us to control the number of theoretical cell-types. The average expression profile of each single-cell in a cluster is used to create the cell-type signature matrix S_{m×k}.

Concentration Matrix

The next task is to solve for the concentration matrix C_{k×n}. Each column of X corresponds to genes measured by qPCR, and is a linear combination of single cell expression profiles with unknown concentrations. Let us denote a particular column in X as x and its corresponding column in C by c. Inferring c can be formulated as the following quadratic program:

$$\begin{matrix}minimize & {‖ Sc - x ‖}_{2} \\ subject to & \sum c = 1 \\ & {c}_{i} \geq 0 ∀ i = 0 … m\end{matrix}$$

This least-squares formulation can be solved with any constrained quadratic programming solver.

Missing Cell Type

Solving for a missing cell type is an extension of the above computational deconvolution framework. The signature matrix including the missing cell type will be denoted as Ŝ_{m×(k+1)}, and the concentration matrix will be denoted Ĉ_{(k+1)×n}. The goal is to simultaneously calculate the missing k^{th} column of signature matrix and the complete concentration matrix. The quadratic formulation is the following:

$$\begin{matrix}minimize & {‖ \hat{S} \hat{C} - X ‖}_{2} \\ subject to & \sum c = 1 \\ & {c}_{i} \geq 0 ∀ i = 0 … m \\ & {s}_{k} \geq 0\end{matrix}$$

This is a non-linear non-negative least squares optimization problem. Damped least-squares (DLS) is used to efficiently solve this problem. The missing cell type signature is initialized as the average of all the known cell types and the concentrations are set uniformly.

One additional challenge is determining if there is a missing cell type present in the mixtures. This is addressed by comparing the residual value of the system with and without the missing cell type, \hat{r}=|X−ŜĈ |, respectively r=|X−SC|. If \hat{r}>r+γ where γ is some small constant then our approach will report that a missing cell type is likely present. The small constant is in place in order to require a non-trivial difference between the two scenarios.

### Simulation

This work relies on two types of data, single cell and bulk data from the same source measured with the same technology. The combination of these data types is not available in any public data sources. Therefore it is necessary to simulate such datasets.

101 cells from 5 cell types (hematopoietic, intestinal, mammary gland, prostate and neural stem cells) were obtained and measured using the Fluidigm C1 and Biomark HD platforms. The 280 genes were chosen using a literature guided approach for known stem cell and lineage specific marker genes. The qPCR data was processed using Fluidigm software, but no normalization to housekeeping genes was performed.

In order to properly access the accuracy of the deconvolution and missing cell type prediction, a semi-continuous model for simulating both single cell and mixture data sets has been adopted. The main motivation behind this model is the bi-modal nature of gene expression observed in single cell experiments, which show that genes are both absent and highly expressed in similar cells. The presented model therefore has two components; the probability of a gene being expressed in a cell, along with the expression value of that gene when it is expressed. The presented model most closely mirrors a semi-continuous model.

Active Transcription

Non-zero Cⁱ_{T} value measurements of a gene i for a particular cell type l are assumed to follow a normal distribution, (μ, σ₂). In the experiments described herein, the parameters of this normal distribution have been estimated from the training single cell data. The random variable is denoted as qⁱₗ.

Expression Probability

The probability that a particular gene is expressed and detected can be estimated directly by counting the number of non-zero expression values for a given gene i and cell type l. The random variable is denoted as pⁱₗ.

Combined Model

These two components can be combined in a variety of ways to create a model for simulating single-cell observations. For a given gene i and cell type l:
1. Wⁱₗ=pⁱₗ·qⁱₗ. Under this model each gene has a certain probably of being expressed, and when it is, the expression level is taken from the normal distribution fitted from the training data available for that gene and that cell type.
2. Wⁱₗ=qⁱₗ. This is a special case of the previous model under which genes are assumed to always be expressed.
3.

$${W}_{l}^{i} = \frac{1 + {p}_{l}^{i}}{2} \cdot {q}_{l}^{i} .$$

In this scenario only the fraction of the expression probability stemming from technical error is represented.

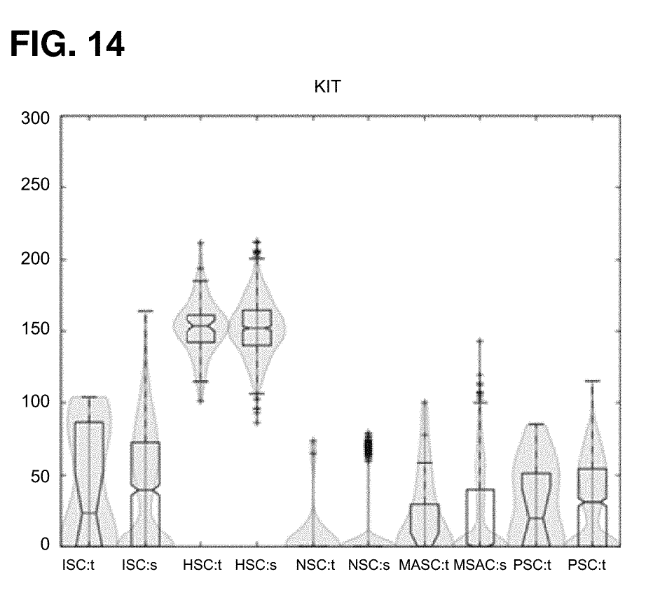

This bi-modal property can be observed by viewing the gene expression distribution of a random gene taken from the starting dataset. The true and simulated distributions are seen side-by-side in FIG. 14.

The procedure for creating simulated single cell for cell type i is:

1. for all j=1 . . . m estimate pⁱⱼ.

2. for all j=1 . . . m estimate the parameters of the normal distribution and sample to obtain qⁱⱼ

3. for all j=1 . . . m multiply pⁱⱼ by qⁱⱼ

Two different scenarios were explored for simulating the concentration matrix C. The first is simply a uniform concentration of all cells. This implies a concentration of ⅕ for this study. The second type was sampling uniformly at random from 0 to 1 exclusive for every cell type, then normalizing by the sum. This ensures the concentration of each cell type in a mixture sums to 1 and there are both high and low concentration cell types.

The mixtures were created by repeatedly sampling single cells according to the proportions dictated by C. The per gene expression levels were added together to create a single mixture. In this simulation, no appropriate housekeeping gene was available for normalization, so the gene expression levels for the mixtures were simply divided by the total number of cells.

An error term is used in the simulations, e=5, 10, 25, 50, 100, 200, which acts as the noise parameter and represents the number of single-cell's used to create a particular mixture. The fewer single cells used, the more different that mixture will be than the sum of cell type signatures.

Unless otherwise stated, each experiment was run 10 times and the presented results are the average of the 10 replicates.

### Results and Discussion

Existing Tools

Several approaches may be used for solving both the supervised and unsupervised gene expression deconvolution problem. The formulation presented here sits between these two versions of the problem. The completely supervised approaches are not able to adequately deconvolve the systems studied here because the canonical cell type signature is required as input. Therefore only the unsupervised methods DSA, Deconf, and the semi-supervised NMF based methods SSKL and FROB are compared against our single cell quadratic programming based method, referred to herein as “UCQP.” The semi-supervised methods are given the opportunity to mine the single cell data to build the descriptive signatures necessary using tools built into the CellMix toolkit.

Experimental Parameters

This work will compare the effect of several experimental parameters across the different deconvolution methods. One of the primary parameters explored is the number of mixtures measured in order to determine the effect of this parameter on accuracy. The error parameter is also varied to compare methods at varying degrees of difficulty, finally the number of genes used in the deconvolution is varied.

Mixture Effects

One important dimension to this single cell aided deconvolution formulation is the number of mixtures necessary to properly deconvolve the system. Since both single cell and bulk qPCR data is necessary it is important that the number of mixtures required be kept small to contain costs. To this end several simulations were carried out where the number of mixtures is varied in the set 5, 10, 15, 20, 25, 30.

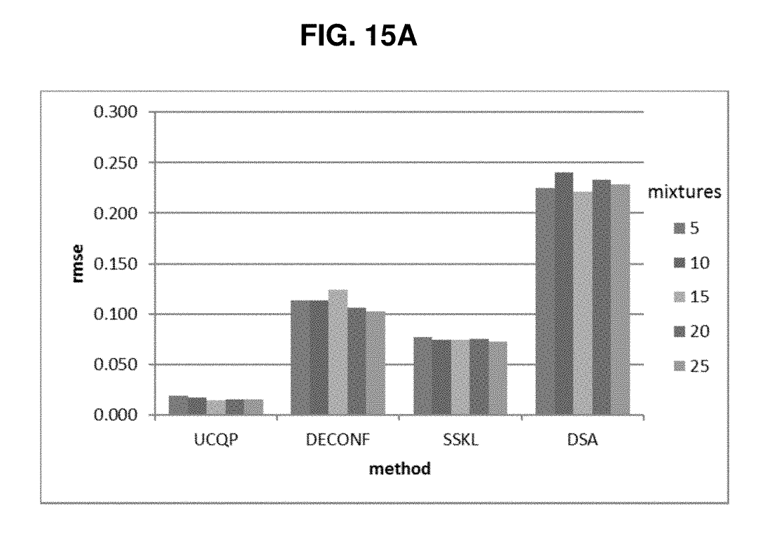

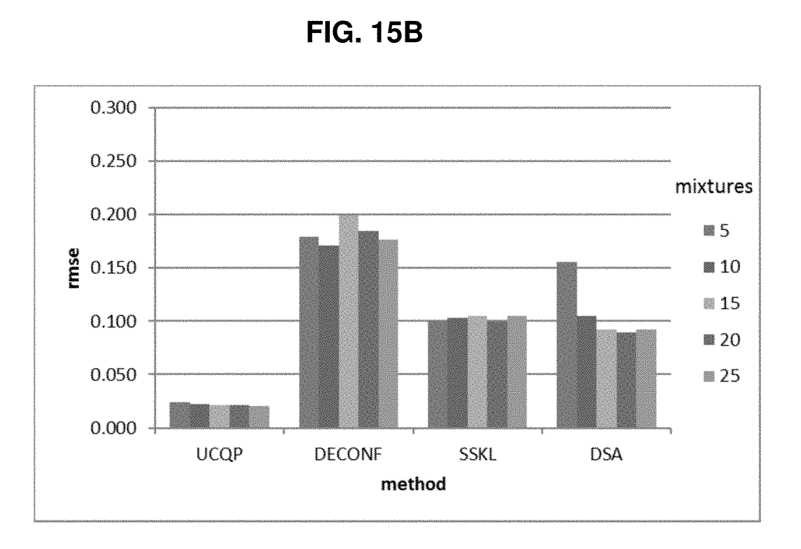

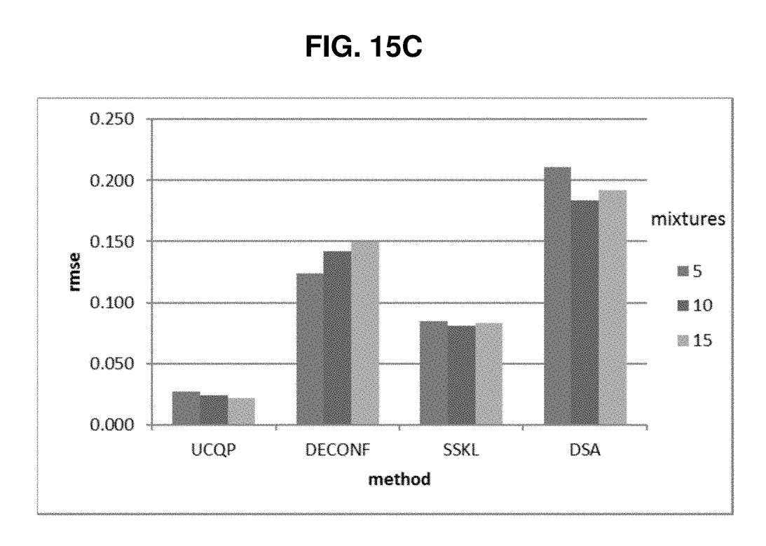

Under the “ideal” scenario seen in FIG. 15A, the number of genes is fixed to 32, the error is set to 40 samples per cell, and the concentration is uniform across all mixtures. There is no absolute pattern or correlation between accuracy as the number of mixtures varies for any of the methods. The UCQP method outperforms both the semi and unsupervised methods.

In FIG. 15B, the error and gene count is at 40 and 32 respectively; however, the concentrations are random. There is again an absence of any connection between rmse as the number of mixture varies. The absolute rmse for DECONF, SSKL suffers slightly, UCQP remains the same and interestingly DSA, the unsupervised method, improves dramatically.

The final comparison in FIG. 15C fixes the error at 40 and the concentrations are uniform. Here the number of genes is halved from 32 to 16. The absolute rmse for all methods seems to suffer little, with a possible anti correlation between mixture count and accuracy observed for DECONF.

The number of mixtures used in deconvolution is a critical parameter as it influences the cost and feasibility of collecting the appropriate amount of data. Finding an algorithm which works well with a minimum number of mixtures necessary makes in silico deconvolution more attractive. The UCQP approach presented here performs deconvolution one mixture at a time and therefore no constraints on the number of mixtures exist. It follows that the accuracy of UCQP should be independent of the number of mixtures. This was observed in FIG. 15. It should also be the case that DECONF, SSKL and DSA all have some dependency on the number of mixtures. However, only a minimal dependency is observed in FIG. 15.

Error Effects

It is important to quantify the ability of the deconvolution algorithm as the level of error changes. In the following simulation experiment the error parameter was varied between 5, 20, 40 and 100.

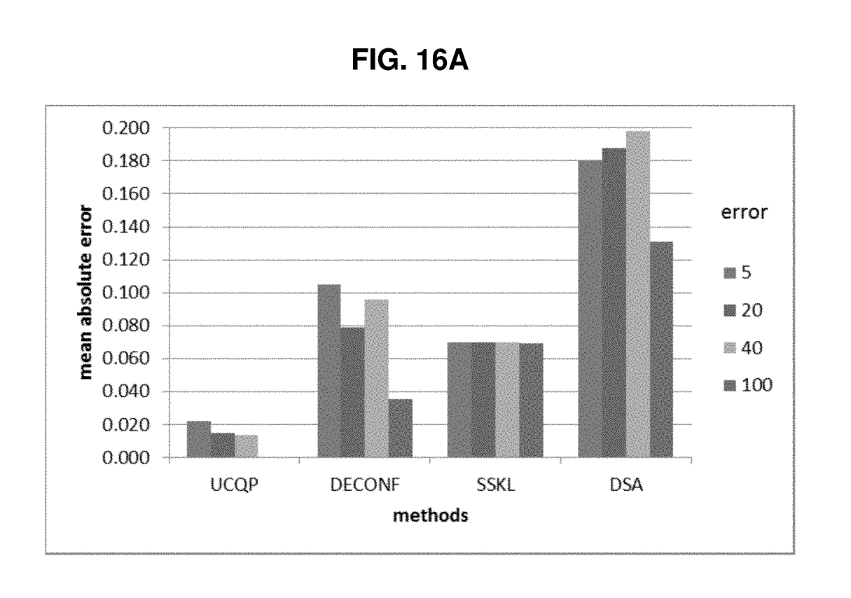

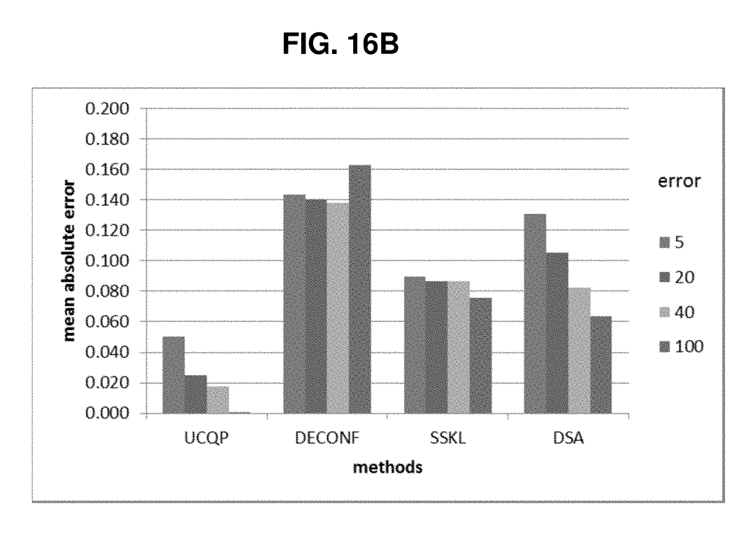

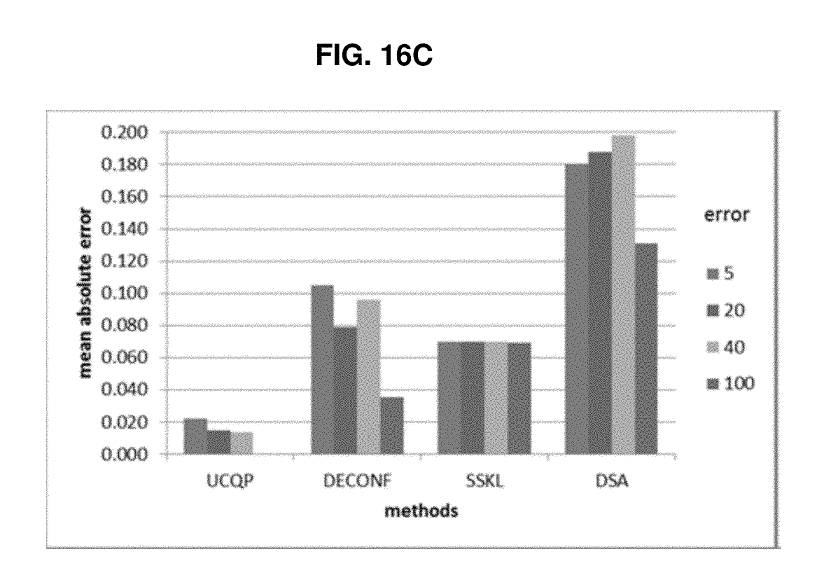

The ideal scenario has the number genes is fixed to 30, there are 10 mixtures and equal concentrations set across cell types. In FIG. 16A the accuracy of UCQP has a direct correlation with the error parameter. At 5 samples per mixture the algorithm performs worst and at 100 it has significantly lower error. The DECONF and DSA algorithms do not seem to have a clear relationship between error and accuracy and SSKL is not effected at all.

In FIG. 16B the number of mixtures is 10, gene count is 32 and the concentration is random. Both the UCQP and DSA algorithms have a direct correlation with the error parameter and rmse. The absolute rmse for UCQP is best for all scenarios.

In the last comparison in FIG. 16C, the number of mixtures and gene count are 10 and 16, respectively, while the concentration is uniform. The results are almost exactly the same as the ideal scenario in FIG. 16A.

The proposed error model is quite simplistic and conflates both technical and biological noise. However without a stronger understanding of both the technical and biological sources a more advanced model may be premature. Adjusting the number of sampled single-cells to create each mixture enables the simulation to create easy scenarios, where each cell type is sampled sufficiently to get close to the canonical average, and more difficult instances where each cell type is far from the average. Additionally since this approach relies on sampling, it will inherently reflect the biological diversity of the supplied single-cells. If each cell population is quite diverse, more sampling will be required to find the average and therefore accurate deconvolution will be more difficult.

The error effects are most dramatic in FIG. 16B where the mixtures consist of random concentrations of each cell type. Intuitively this scenario seems the most challenging with respect to inferring cell type concentration and it seems natural that increased error makes the problem more challenging. Both UCQP and DCA improve as the sampling rate is increased, while the NMF methods do not respond as clearly. No explanation for these effect is immediately apparent. One advantage of the simpler quadratic programming based method is that understanding effects becomes easier.

Gene Effects

The number of genes necessary to measure is an important parameter for this approach. Therefore a wide range of gene counts were surveyed in order to assess the effect across a variety of methods for qPCR. Values of 8, 16, 32, 64, 128 and 256 were tested. The genes were not selected at random, but rather were ranked using ANOVA test, excluding the lower scoring genes first.

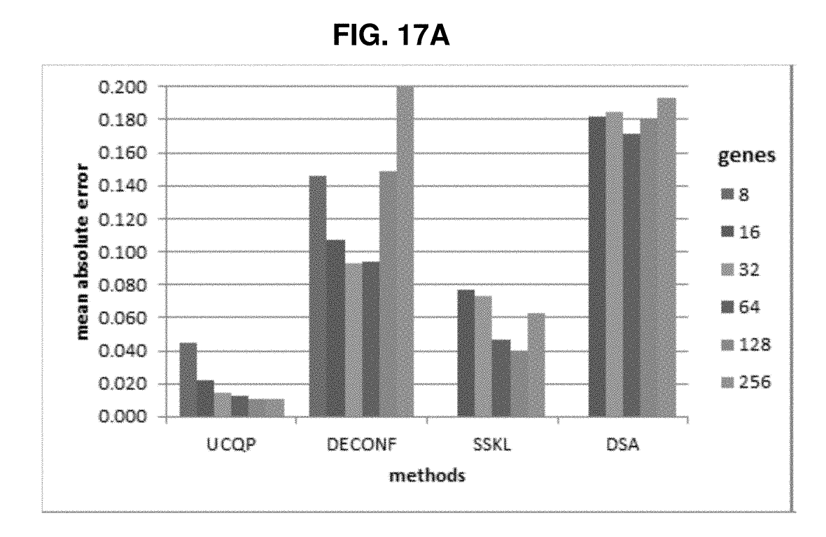

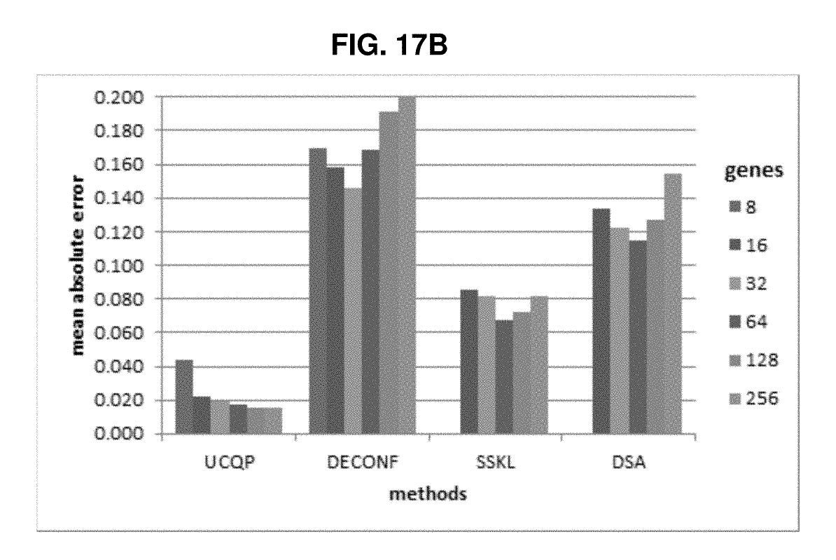

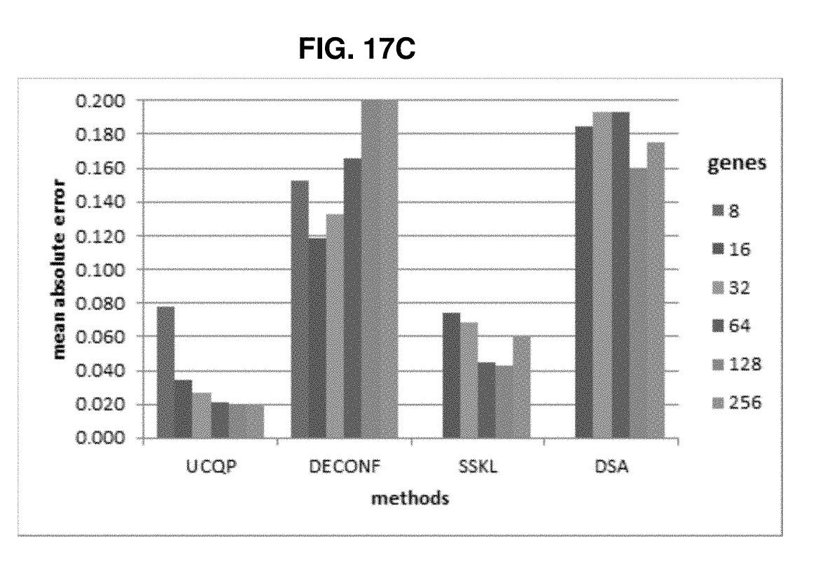

In the ideal scenario depicted in FIG. 17A, the error parameter is 40, concentrations are uniform and the number of mixtures is fixed at 5. Here, UCQP outperforms all methods in terms of mean absolute error. There is a clear inverse relationship between the number of genes used and error in UCQP. No other method has this clear relationship.

FIG. 17B has the error fixed at 40, mixtures 5 and the concentrations are random. For UCQP this more challenging scenario has no noticeable effect on mean absolute error. There is still a clear inverse relationship between gene count and error for UCQP and no relationship for other methods.

The final scenario in FIG. 17C fixes the number of mixtures to 5, the concentrations are uniform but the error parameter is set to 5. Here the high error rate clearly changes the absolute error of UCQP, however the inverse relationship between gene count and error rate still exists.

When considering just the UCQP method in FIG. 17, the effect of number of genes is quite clear. There exists a saturation point where the accuracy of the estimates no longer improves when adding more genes. This behavior is not observed in any of the other approaches. It should be noted that this simulation does not start with a random subset of genes, in fact all genes used in the data behind the simulation were chosen by domain experts. Also it is interesting to note that a high degree of accuracy can be obtained by using as few as 16 genes.

Missing Cell Type

Simultaneously estimating the canonical gene expression signature for a missing cell type and the concentrations of all cells is a difficult challenge. In order to test the proposed non-linear optimization approach a basic leave-one-out scenario was utilized. In this testing setup, each cell type was left out and its signature was estimated using the presented approach (UCQPM) compared against DECONF which can be told to estimate all cell types. As before, this scenario was repeated 10 times and the following results are the average of the 10 replicates. A second study our basic model used to detect if a cell type is missing or not. This was presented as a binary classification problem and assessed accordingly.

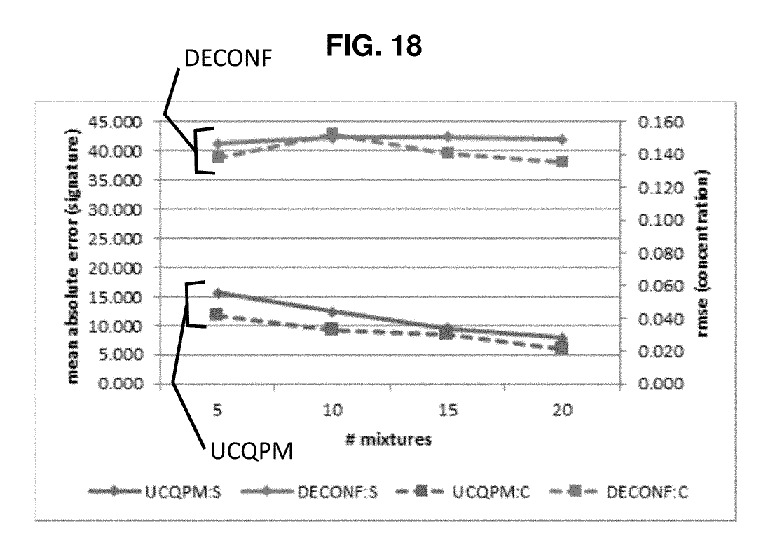

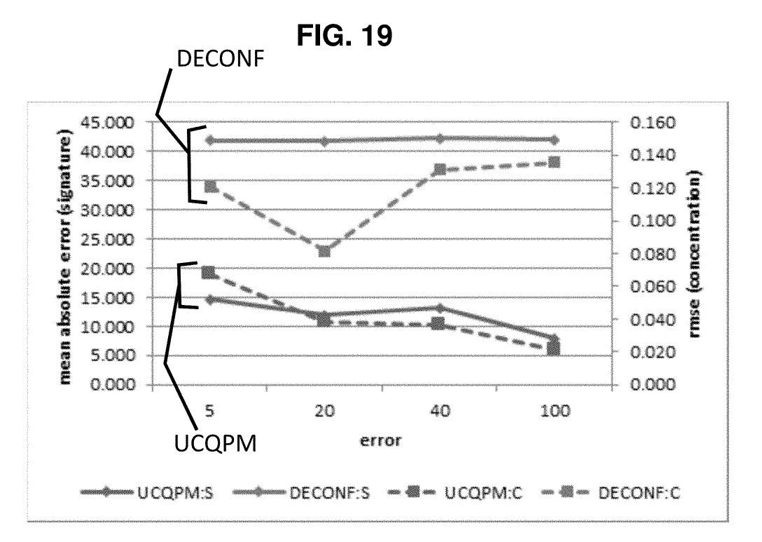

In general the UCQPM method outperforms unsupervised deconvolution on all of these missing cell type experiments. The results for these experiments are detailed in FIGS. 18 and 19. This holds for all tested conditions.

In FIG. 18, the mixture effect is measured. There is an inverse relationship between both the concentration and signature estimates and error for UCQMP. This is consistent with results observed in the non-missing scenario. For DECONF no such consistent relationship is observed.

FIG. 19 details the effect of noise on missing cell type estimates. The number of mixtures is fixed to 20, the concentration is random, and 64 genes are used. For UCQPM there is a clear inverse relationship between error and accuracy. At a high error rate (5 cells per type), both the signatures and concentrations are estimated poorly. As the error parameter increases the mean absolute error and rmse decreases which is also reflected in the non-missing scenario.

The final test evaluates our basic predictor of a missing cell type. The algorithm applies both missing and non-missing approaches to a given deconvolution problem, then applies the basic test \hat{r}>r+γ where γ is some small constant. Where \hat{r} is the residual calculated assuming a missing cell type, r is without a missing cell type. This scenario was tested by creating 10 mixtures with a missing cell type and 10 without. Both UCQPM and UCQP were run on each dataset and the test was applied predicting if it contained a missing cell type. This whole scenario was repeated 10 times with the average results displayed in Table 1.

TABLE 1
This table records the binary classification problem attempting to
predict the presence of a missing cell type from single cell and bulk
qPCR data. There error column indicates the error parameter used in
the simulation (higher being lower noise). The accuracy column is the
typical notion of accuracy, and AUC is the area-under-curve.

| Error | Accuracy | AUC |
|---|---|---|
| 70 | 0.77 | 0.89 |
| 80 | 0.72 | 0.86 |
| 90 | 0.72 | 0.84 |
| 100 | 0.77 | 0.81 |

In general, this leave-one-out experiments indicates that UCQPM provides a more robust estimate of the missing cell type signature and concentrations than the unsupervised approach. This result is expected because the UCQPM utilizes more information about the system and is estimating a smaller number of variables. The quadratic programming formulation allows the method to be tailored to any number of unique situations, maximizing the use of available information.

### CONCLUSIONS

This work presented an in silico gene expression deconvolution and missing cell type detection algorithm which is based on quadratic programming. This supervised approach relies on single cell resolution data to estimate cell type signatures, then uses a well-defined, non-negative least squares objective to estimate cell type concentrations. This approach was compared against leading semi-supervised and unsupervised NMF based methods and was shown to perform well. Estimating a missing cell type signature is also possible using a basic quadratic programming formulation. The signature and concentration estimation accuracy is much better than completely unsupervised NMF based methods.

### Other Embodiments

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. A method of determining the gene expression profile of a rare cell type in a population of cells, said method comprising:
a. providing gene expression profiles of a plurality of individual cells in said population;
b. using the gene expression profiles provided in (a) to determine a predicted gene expression profile of the entirety of said population; and
c. minimizing the difference between the predicted gene expression profile determined in (b) and an experimentally determined gene expression profile of the entirety of said population;
wherein said minimizing determines the gene expression profile of said rare cell type.

2. The method of claim 1, wherein the predicted gene expression profile of the entirety of said population is determined using predicted gene expression profiles of a plurality of cell types present within said population and a calculated cell type composition of said population.

3. The method of claim 2, wherein the predicted gene expression profiles of said plurality of cell types present within said population are determined by grouping the gene expression profiles of said plurality of individual cells into like clusters.

4. The method of claim 3, wherein said grouping comprises performing principle component analysis (PCA); and/or
wherein said grouping comprises performing K-means clustering.

5. (canceled)

6. The method of claim 1, wherein said difference between the experimentally determined gene expression profile of the entirety of said population of cells and the predicted gene expression profile of the entirety of said population of cells is minimized by least-squares optimization.

7. The method of claim 1, wherein the gene expression profiles of said plurality of individual cells in said population are determined by measuring the abundance of one or more RNA transcripts, or DNA equivalents thereof, in each of said individual cells.

8. The method of claim 1, wherein the experimentally determined gene expression profile of the entirety of said population of cells is determined by measuring the abundance of one or more RNA transcripts, or DNA equivalents thereof, in the entirety of said population.

9. The method of claim 7, wherein the abundance of said one or more RNA transcripts, or said DNA equivalents thereof, is measured using a polymerase chain reaction (PCR) assay.

10. The method of claim 9, wherein said PCR assay is a quantitative reverse transcription PCR (qRT-PCR) assay; and/or
wherein said PCR assay is conducted by contacting DNA or RNA isolated from said individual cells with an oligonucleotide complementary to a portion of a gene of interest.

11. (canceled)

12. The method of claim 10, wherein said oligonucleotide comprises a radioisotope, fluorescent compound, bioluminescent compound, a chemiluminescent compound, metal chelator, or enzyme; and/or
wherein said oligonucleotide comprises a fluorescent compound; and/or
wherein said oligonucleotide further comprises a fluorescence quencher.

13-14. (canceled)

15. The method of claim 7, wherein the abundance of said one or more RNA transcripts is measured using a RNA sequencing (RNA-Seq) assay.

16. The method of claim 1, wherein the gene expression profiles of said plurality of individual cells in said population are determined by measuring the abundance of one or more proteins in said individual cells.

17. The method of claim 1, wherein the experimentally determined gene expression profile of the entirety of said population of cells is determined by measuring the abundance of one or more proteins in the entirety of said population.

18. The method of claim 16, wherein the abundance of said one or more proteins is measured using an immunohistochemical assay, a western blot analysis, immunoprecipitation, an enzyme-linked immunosorbent assay (ELISA), an enzyme-linked immunofiltration assay (ELIFA), a molecular binding assay, mass spectrometry, mass spectrometric immunoassay, or a biochemical enzymatic activity assay.

19. The method of claim 1, wherein said rare cell type is present within said population of cells at low abundance; and/or
wherein the rare cell type is present within the population of cells at concentrations below the limit of detection of a gene expression assay.

20. (canceled)

21. The method of claim 19, wherein said gene expression assay is selected from the group consisting of PCR, qRT-PCR, RNA-Seq, an immunohistochemical assay, a western blot analysis, immunoprecipitation, ELISA, ELIFA, a molecular binding assay, mass spectrometry, mass spectrometric immunoassay, and a biochemical enzymatic activity assay.

22. The method of claim 1, wherein the rare cell type comprises a component of a mixture of cells; and/or
wherein the rare cell type comprises adult cells and/or cancer cells.

23. (canceled)

24. The method of claim 22, wherein the cancer cells comprise circulating tumor cells and/or rare cancer cells; optionally
wherein the rare cancer cells are present in a tissue biopsy.

25. (canceled)

26. The method of claim 1, wherein said rare cell type is selected from the group consisting of a hematopoietic, intestinal, mammary gland, prostate, and neural stem cell.

27. A method of identifying the cell type composition of a population of cells, said method comprising:
a. providing gene expression profiles of a plurality of individual cells in said population;
b. using the gene expression profiles provided in (a) to determine predicted gene expression profiles of a plurality of cell types present within said population by grouping the gene expression profiles of said plurality of individual cells into like clusters; and
c. comparing the gene expression profiles determined in (b) with the gene expression profile of the entirety of said population of cells;
wherein said comparing identifies the cell type composition of said population of cells.
